(19) United States
(12) Patent Application Publication
(10) Pub. No.: US 2017/0050021 A1
(43) Pub. Date: Feb. 23, 2017

(54) Random pulsed high frequency therapy

(71) Applicant: Eric Richard Cosman, SR., Belmont, MA (US)

(72) Inventor: Eric Richard Cosman, SR., Belmont, MA (US)

(21) Appl. No.: 10/922,173

(22) Filed: Aug. 20, 2004

(51) Int. Cl. A61N 1/36; A61N 1/06; A61N 1/40; A61N 1/05

(52) U.S. Cl. CPC A61N 1/36017; A61N 1/0551; A61N 1/0529; A61N 1/36021; A61N 1/36025; A61N 1/06; A61N 1/403

(57) ABSTRACT

A method and apparatus for modifying a function of tissue can include the application of an electromagnetic signal output to tissue cells. The electromagnetic signal output can have a waveform with bursts of signal output amplitude that produces fields in the tissue during the burst on-time that can modify cell components. The on-time periods are followed by off-time periods of low signal output amplitude which produce lesser modification effects on cell components. The on-time periods have time durations that are non-predetermined and random. The rate of on-time bursts can be regular, and in one example periodic. In one example, the waveform has amplitudes during the on-time periods and durations of on-time periods that causes elevation of the temperature of tissue that exceeds the lethal temperature levels of 45 to 50° C.

### TECHNICAL FIELD

This invention relates generally to field therapy.

### BACKGROUND

The use of radiofrequency (rf) generators and electrodes to be applied near or in neural tissue for pain relief or functional modification is well known. For instance, the RFG-3C RF Lesion Generator of Radionics, Inc., Burlington, Mass., and its associated electrodes enable placement of the electrode near neural tissue and heating of that tissue by rf resistive power dissipation of the generator power in the tissue. Thermal monitoring by a thermo sensor in the electrode has been used to control the process. Heat lesions with tissue temperatures of 60 to 95 degrees Celsius (° C.) are common. Tissue dies by heating at about 45 to 50° C., so this process is a heat lesion generation and is designed to elevate the neural tissue above this lethal temperature threshold. Often, the procedure of heating above 45 to 50° C. causes severe pain to the patient which is so unpleasant and frequently unbearable that local or general anesthetic is required during the heat procedure. Use of such anesthetics has a degree of undesired risk to the patient, and the destructive nature of and unpleasant side effects of the rf heat lesion are limitations of this technique, which is well known. Heat lesion generators typically use continuous wave rf generators with radiofrequencies of between 100 Kilohertz to several Megahertz (viz. the rf generators of Radionics, Fischer, OWL, Elekta, Medtronic, Osypka, EPT companies). The theory and use of rf lesion generators and electrodes for pain and functional disorders is described in various papers; specifically see: (1) Cosman, et al. “Theoretical Aspects of Radiofrequency lesions and the Dorsal Root Entry Zone.” Neurosurg 15:945-950, 1984; and (2) Cosman ER and Cosman BJ. “Methods of Making Nervous System Lesions,” in Wilkins R H, Rengachary SS (eds): Neurosurgery. New York, McGraw-Hill, Vol. III, 2490-2498; and are hereby incorporated by reference herein in their entirety.

Neural stimulation is also now a common method of pain therapy. Stimulus generators with outputs of 0 to 10 volts (or zero to several milliamperes of current are used) are typical. This refers to the outputs of stimulators of various manufacturers which are used to produce a physiologic response or change in physiological function. The frequency ranges in Hertz (Hz) of these stimulators refers to the pulse repetition frequency of the stimulator pulse outputs. The description of the output parameters of such pulse trains can be described as the pulse rate in units of Hz or more definitively, the number of pulses per second (pps) of the output. The actual frequency spectrum of a train of stimulation output pulses with, for example, a pulse repetition rate of 150 Hz, or more clearly 150 Hz (pps), can actually have significant and dominant frequency components well into the KiloHertz (KHz) frequency range with relatively insignificant frequency components at 150 Hz or even below 1 KHz. The “physiologic frequency range” of a neural stimulator, which means the range of pulse repetition rates (typically designated in Hertz (pps) units) of the stimulator's output signal is significantly different from the range of frequencies of the frequency components, the sinusoidal oscillating signals, which constitute the Fourier spectrum of the stimulator output signal. Each frequency component of the frequency spectrum of the stimulator output is also characterized by a frequency value designated in Hertz (Hz) units, however, the frequency value in Hz of the most intense frequency components can be very different from the stimulator output pulse repetition rate or, as it is sometimes called, the pulse repetition frequency in Hz (pps).

Signal output from neural stimulators is typically delivered to electrodes placed near or in neural tissue on a temporary basis (acute electrode placement) or permanent basis (chronic electrode implants). Such stimulation can relieve pain, modify neural function, and treat movement disorders. Typically, the stimulation is applied for a long period of time or reapplied repeatedly for a long time in order to have a long-term effect, i.e., usually when the stimulus is turned off, the pain will return or the therapeutic neural modification will cease after a short time (hours or days). Thus permanent implant electrodes and stimulators (battery or induction driven) is standard practice (viz. see the commercial systems by Medtronic, Inc., Minneapolis, Minn.), and the stimulus is usually sustained or repeated on an essentially continuous basis for years to suppress pain or to treat movement disorders (viz. Parkinsonism, bladder control, spasticity, etc.). Stimulators deliver regular pulse trains or repetitive bursts of pulses in the range of 0 to 200 Hertz (pps) or somewhat higher (i.e., a physiologic range similar to the body's neural frequency pulse rates), so this method simulates or inhibits neural function at relatively low frequency. It does not seek to heat the neural tissue for destructive purposes as in high frequency technique. Chronically or permanently implanted stimulators often require battery changes or long-term maintenance and patient follow-up, which is expensive and inconvenient, often requiring repeated surgery.

One example of a neural stimulator is the Activa stimulator system, a commercial product from Medtronics, Inc. of Minneapolis, Minn. It is an implantable stimulator and electrode system to stimulate the brain of humans to control symptoms of Parkinson's disease. The Activa brochure from Medtronic, Inc. UC9605628EE NI-2854EE of 1997 describes the Itrel II Implantable Pulse Generator (IPG) as having selectable stimulation output parameter ranges of: pulse amplitude, 0 to 10.5 volts; pulse rate, 2 to 185 Hz (pps); and pulse width 60 to 450 microseconds (μsec). Pulse width can also be referred to as pulse duration.

Another example is the X-TREL stimulator for use in the spinal cord to treat pain. This is described in Medtronic document 195196-017 of April 1989. It has output signal parameter ranges of: pulse amplitude, 0 to 10 volts; pulse rate, LO range of 0 to 120 Hz (pps), and HI range of 0 to 1400 Hz (pps); and pulse width, 50 to 200 microseconds for the HI pulse rate range, and 50 to 1000 microseconds for the LO pulse rate range. Monophasic or biphasic pulse waveform outputs are selectable.

Most modern radiofrequency (rf) lesion generators that are used to treat neurological diseases with rf energy that is delivered through electrodes inserted into or placed into the body also contain a neural stimulator the signal output of which when delivered to the electrode in the body is designed to produce a physiologic stimulation response such as motor and sensation reaction by the nerves. In an example, the RFG-3CF and RFG-3C PLUS rf generators of Radionics, Inc. (Burlington, Mass.) have stimulation output parameter ranges of: amplitude of 0 to 1 and 0 to 10 volts; pulse rate of 2 to 200 Hz (pps); and pulse duration of 0.1 to 1 millisecond (ms) (equivalent to 100 to 1000 μsec). The pulse waveform is a series of biphasic pulses being repeated at a frequency rate of 2 to 200 pps. Each biphasic pulse is approximately a negative square pulse followed immediately by a positive square pulse, each of the pulses having the same amplitude in volts, and each having a square width equal to the selected pulse duration. The rf generators are described in Radionics brochures 914-91-001 Rev. A. and 915-91-001 Rev. A of 1999.

In another example, the rf generator Model N50 of Leibinger GmbH has stimulation ranges for its biphasic pulse output of: amplitude 10 to 10 volts; pulse rate 1 to 200 Hz (pps); and duration 0.5 to 5 ms. It is described in the Leibinger brochure 90-05400 of 1994.

The distribution and range of frequencies of the oscillatory or sinusoidal frequency components which make up a stimulator output signal that is within the “physiological stimulation frequency range” (PSFR) of a neural stimulator can be derived from a Fourier transform or a Fourier spectral analysis of the stimulator output. This can reveal the range of dominant and of significant frequency components in the physiologic stimulator signal. A description of the Fourier transform analysis and the derivation of the frequency component spectrum of pulsatory signals, similar to a stimulator signal, is given in the textbook Information Transmission, Modulation, and Noise by Mischa Schwartz, McGraw-Hill Book Company, New York, second edition, 1970. Another article entitled “Electric and Magnetic Fields for Bone and Soft Tissue Repair” by Charles Polk in the Handbook of Biological Effects of Electromagnetic Fields, pp. 231 to 246, edited by Charles Polk and Elliot Postow, second edition, CRC Press LLC, 1996, stresses the difference between the pulse repetition frequency of a signal and the frequency content of the signal in terms of Fourier integral analysis, and this article is hereby incorporated by reference herein in its entirety.

In one example of a Fourier frequency component analysis of a stimulation signal output which is in the PSFR, the Radionics Model RFG-3CF rf lesion generator has a built-in nerve stimulator which outputs biphasic pulses which can be selected to have pulse rates of 150 Hz (pps) and pulse durations of 0.1 millisecond. The Fourier transform of this signal shows the frequency spectrum composition of this signal to have frequency components over a wide range. The amplitudes of the frequency components vary in lobe-like variations versus frequency. The first maximum, or lobe, has the largest amplitude in the component spectrum and occurs between about 2.5 to 7.5 kilohertz (KHz). The second maximum or lobe occurs between about 12 and 17 KH3, and its amplitude is about 29 percent of the first maximum lobe. The third lobe maximum occurs between 22 and 27 KHz, and its amplitude is about 18 percent of the first maximum. The fourth maximum is between 32 and 37 KHz, and its amplitude is about 13 percent of the first maximum. The fifth maximum is between 42 and 47 KHz, and its amplitude is about 10 percent of the first maximum. The frequency components continue to even high frequencies, but continue to weaken with increasing frequency. The Fourier amplitude at zero frequency is zero. The amplitude at the so-called stimulator pulse “frequency” of 150 Hz (pps) is only about 6 percent of the maximum amplitude at about 4 KHz, showing that the pulse repetition frequency should not be confused with the actual oscillatory frequency components. The frequency components of significance within the PSFR, which includes this stimulator signal having a so-called frequency of 150 Hz (meaning pulses per second (pps)), are very far away in frequencies from 150 Hz.

In another example that illustrates a stimulator output in the “physiologic stimulation frequency range” and the distribution in amplitude and oscillating frequency of continuous sinusoidal signal components which make up the stimulator output, the Medtronic X-TREL stimulator can produce stimulation signals with biphasic pulse trains having a pulse repetition rate frequency of 120 Hz (pps) and pulse width of 50 microseconds. This output signal is composed of significant oscillating frequency components distributed over a broad frequency range. The first and largest amplitude group of frequency components occurs around 5 to 12 KHz, a secondary group of frequency components occurs around 25 to 35 KHz with amplitudes of about 30 percent of the first group, a third group of components occurs at around 42 to 57 KHz with amplitudes of about 20 percent of the first group, a fourth group occurs around 62 to 72 KHz with amplitude of about 13 percent of the first group, a fifth group occurs at about 82 to 95 KHz at about 10% strength of the first group. Further groups of components occur at higher frequencies than 100 KHz but have weaker strengths. Frequency components near zero Hz have about zero strength, and the frequencies components near 120 Hz (the pulse rate “frequency”) have strength about 1 percent of the amplitude of components near the largest maximum first group near 5 to 12 KHz.

From the examples of the RFG-3CF and X-TREL, the commonly used physiologic stimulators with their commonly used output parameters with pulse widths of 50 to 100 microseconds produce predominant frequency components in the 1 to 20 KHz range. Secondary groups of frequency components with amplitudes of about 30% of the predominant components occur in the 10 to 40 KHz range. Lesser amplitude components of 10 to 20 percent of the predominant components amplitudes occur in the 40 to 80 Hz range. Above about 85 to 95 KHz, the frequency component's amplitudes are weaker and typically less than 10 percent of the dominant component's amplitudes. Thus the PSFR includes sine-wave frequency components in the 0 to 10 KHz and the 10 to 20 KHz ranges as predominant factors, and includes lesser but still significant components up to about 40 KHz. The PSFR also includes components in the 40 to 80 KHz range having weaker amplitudes. It also includes very weak components in the 85 to 90 KHz range. From 100 KHz and above, the components are extremely weak and can be considered to be not included in the PSFR.

In an article entitled “Sensory response elicited by sub cortical high frequency electrical stimulation in man” by W. W. Alberts, et al., J. Neurosurg., Vol. 36, pages 80 to 82, 1972, the sensory response of sine-wave signals applied to the thalamus of the brain was studied. This article is hereby incorporated by reference herein in its entirety. The stimulus was applied through an electrode in the thalamus, and sine-wave signals with frequencies from 0.1 to 250 KHz were applied. The amplitude threshold, in amplitude of voltage and current, of the sine-wave to achieve a sensory response was low in the 0.1 to 10 KHz range, but increased rapidly from 10 KHz to 100 KHz. Some sensation was felt at 100 KHz, but this required a threshold amplitude of 25 to 100 times the threshold amplitudes below 10 KHz. This can result from the neural response at 0 to 10 KHz being much more effective than at 100 KHz. The response threshold at 50 KHz is about one third that at 100 KHz, which can result from the stimulative response at 50 KHz being much more effective than at 100 KHz. Frequency components in the 0 to 10 KHz and 10 to 50 KHz range produce significant sensory response, but at 100 KHz the sensory response is less effective as gauged by the much higher threshold amplitude required at 100 KHz. An effective stimulation range of sinusoidal frequency components is 0 to 50 KHz, which is consistent with the PSFR described above in connection with common effective stimulation parameters.

Electrosurgical generators have been in common use for decades cutting and coagulating tissue in surgery. They typically have a high frequency, high power generator connected to an electrode that delivers a high power output to explode tissue for tissue cutting and to cook, sear, and coagulate tissue to stop bleeding. Examples are the generators of Codman, Inc., Randolph, Mass., Valley Labs, Inc. Boulder, Colo., and EMC Industries, Montrouge, France. Such generators have high frequency output waveforms which are either continuous waves or interrupted or modulated waves with power controls and duty cycles at high levels so that tissue at the electrode is shattered and macroscopically separated (in cutting mode) or heated to very high temperatures, often above cell boiling (100° C.) and charring levels (in coagulation or cauterizing mode). The purpose of electrosurgery generators is surgical, not therapeutic, and accordingly the output controls, power range, duty cycle, waveforms, and monitoring is not designed for gentle, therapeutic, neuro-modulating, sub-lethal temperature application. Use of an electrosurgical unit requires local or general anesthetic because of its violent and high-temperature effect on tissues.

The inventors M. E. Sluijter, W. J. Rittman, III, and E. R. Cosman have the following patents: U.S. Pat. No. 5,983,141, issued Nov. 9, 1999, entitled “METHOD AND APPARATUS FOR ALTERING NEURAL TISSUE FUNCTION”; U.S. Pat. No. 6,161,048, issued Dec. 12, 2000, entitled “METHOD AND SYSTEM FOR NEURAL TISSUE MODIFICATION”; and, U.S. Pat. No. 6,259,952 B1, issued Jul. 10, 2001, entitled “METHOD AND APPARATUS FOR ALTERING TISSUE FUNCTION.” These four patents are incorporated by reference in their entirety.

The four above-referenced patents are directed at altering the function of neural tissue in a patient. An electromagnetic signal is applied to neural tissue through an electrode. The signal has a frequency component above the PSFR and an intensity sufficient to produce an alteration of neural tissue, and a waveform that prevents lethal temperature elevation. The BACKGROUND section of the patents refer to neural stimulators such as from Medtronic having waveforms and pulse trains in the physiologic frequency range of about 0 to 300 Hz, and they refer to stimulators delivering regular pulse trains or repetitive bursts of pulses in the range of 0 to 200 Hertz (i.e., a physiologic range similar to the body's neural frequency pulse rates). The embodiments described in the patents have signal generator outputs with waveforms having bursts of a high frequency component above the physiologic stimulation frequency range. They describe that the high frequency of the high frequency component “may also range up into the radiofrequency or microwave range (viz. 50 Kilo Hertz to many Mega Hertz).”

The four above-mentioned patents are directed at an electrical signal applied to neural tissue having a waveform with bursts of a radiofrequency signal. The waveform bursts have at least one frequency above the physiologic frequency range. The waveform has predetermined time periods of on-time bursts of rf output of a first predetermined duration, and each burst if followed by an off-time period of a second predetermined duration.

The four above-mentioned patents are directed at application of an electrical signal to neural tissue, the signal having a waveform having an amplitude modulated signal with at least one frequency component above the physiological stimulation frequency range, the signal producing an alteration of the function of the neural tissue corresponding to non-lethal temperature elevations that are less than about 45 to 50° C.

The four above-mentioned patents are directed at an electrical signal applied to neural tissue having a waveform with bursts of a radiofrequency signal which has a frequency component above the physiological stimulation range. Each burst has a predetermined on-time of a first predetermined time duration followed by an off-time output period of a second predetermined time duration.

The above-mentioned four patents are directed at application of an electrical signal to neural tissue by an electrode which has interrupted rf waveforms with bursts having predetermined on-times and predetermined off-times so that the ratio of on-time duration to off-time duration is approximately two percent. The waveform has a frequency component above the PSFR.

The above-mentioned four patents are directed at systems and methods to produce predetermined durations of on-time bursts and predetermined durations of off-time periods of the bursts, and/or to produce predetermined ratios of the on-time and off-time durations with a determined value of the ratio. Electronic circuitry can produce timing signals and switching devices that can be used to produce on-time and off-time signal output waveforms. Predetermined durations of timing switching of electronic circuits has some degree of error caused by limitations of specifications of circuit components, noise, electronic jitter, and other physical factors. Typically, errors of these factors can be made less than 1 to 2% in modern electronics. For example, to produce an electronic burst of a predetermined on-time of 20 milliseconds of rf output signal, the variation of the on-time durations can be made with ordinary electronics to be in the range of 20 milliseconds plus or minus 0.2 milliseconds. In another aspect, a predetermined ratio of on-time to off-time durations of output signal of approximately 2 percent can readily be achieved by circuits to produce ratio values of 2 percent plus or minus 0.02 percent.

### SUMMARY

In general, the present invention includes a generator of an electrical signal output adapted to apply a signal output to tissue in the living body through a signal applicator, the signal output having a waveform corresponding to bursts of output signal, the bursts having non-predetermined time durations of on-time bursts. In one example, the non-predetermined on-time durations can be random in time duration. In another example, the time durations of burst on-times can be non-predetermined and have an average value of duration that can be approximately a selected value, and the variation of time-durations around the average value can cover a significant range relative to the average value. In another example, the rate of on-time bursts can be regular; or can be periodic; or can be constant; or can be predetermined according to a known function of time according to clinical needs. In another example, the bursts of output signal can have non-predetermined on-time durations, corresponding to periods when the output signal amplitude has substantial effect on the tissue followed by periods of non-predetermined off-times during which the output signal amplitude is substantially lower than the on-time signal amplitude so as to have a substantially lesser effect on the tissue it is applied to.

In one aspect, a method for achieving modification of bodily tissue includes applying an output signal from a generator to the tissue by a signal applicator, the signal output having a waveform having bursts of on-time signal amplitude that are non-predetermined in time duration. In one example, the non-predetermined on-time bursts occur at a repetition rate, corresponding to the time between the beginning of the on-time bursts, that is a commencement time that: in one example is regular; in one example is constant; in one example is periodic; in one example is according to a known function of time; in one example is predetermined; and in one example is predictable.

In another aspect, the ratio of on-time signal burst durations to off-time signal burst durations is non-predetermined. In one example, the average value of that ratio can be a predetermined value, and the variation of the ratio around the average value of the ratio can be substantial. In one example, the variation can be: greater than 2%; or greater than 10%; or greater than 20%; or greater than 50%; or greater than 70%; or more of the average value.

An advantage is that non-predictable on-time burst durations can produce tissue modifying variations of the tissue when the output signal is applied to the tissue so that an average level of tissue modification is achieved without sustained extreme output amplitude exposure to the tissue.

Unpredictable, and/or random, and/or non-predictable variations of the time durations of the on-time periods of bursts of output signal can produce corresponding non-predictable variations of heat flashes, or heat bursts, in the tissue near the signal applicator. In one example, the on-time durations and waveform amplitudes correspond to heat burst in the tissue corresponding to temperatures that are greater than the lethal temperature levels of 45 to 50° C. In another example, the on-time durations and signal amplitudes correspond to heat burst temperatures that are below the lethal temperature levels of 45 to 50° C. In one aspect, non-predetermined variations of on-time burst durations produce non-predetermined levels and durations of temperature burst on the tissue.

One advantage is that non-predetermined, and/or random, and/or non-predictable variations of burst temperature durations and/or burst temperature amplitudes can: modify; and/or kill cells; and/or cell bio-structures; and/or bio-molecules of a targeted type over a larger volume and/or over a wider range of types of cells, and/or bio-structures, and/or bio-molecules without causing sustained damage to more temperature resistant non-targeted cells, and/or bio-structures, and/or bio-molecules.

Another advantage is that effects of electric, and/or currents, and/or field gradient fields caused by bursts of varying and non-predictable, random, or non-predetermined duration can cause corresponding variations in duration-dependent effects on bio-structures and spread the modification effect on these bio-structures over a wider range of bio-structures and/or a wider volume of targeted cells. Non-predetermined burst durations corresponding to non-predetermined variations in recovery periods for modification effects on bio-structures can prevent adaption of the targeted structures from fully recovering from the modification effect, thus increasing the effectiveness of the treatment.

In one aspect, the signal output can have a waveform with frequency components within the PSFR that can cause modifying effects on targeted cells and their substructures

In another aspect, the output waveform can have frequency components above the PSFR to produce desired modifying effects. In one aspect, the frequency components can be in the rf frequency range.

In one aspect, the application of signal output can be used to treat: pain; or movement disorders; or neural structures; or neural-muscular structures; or epilepsy; or mood disorders.

One advantage is that the system and method can be used painlessly and easily, avoiding usual discomforts of standard rf heating procedures, yet relief of the pain or the neural disfunction (such as for example motor disfunction, spasticity, Parkinsonism, tremors, mood disorders, incontinence, etc.) can be long lasting using the novel system of the present invention, giving results in many cases that are comparable to those of rf heat lesions done at much higher temperatures. Some applications of this invention may include such examples as relief of back, head, and facial pain by procedures such as dorsal root ganglion or trigeminal ganglion treatments, spinal cord application for relief of intractable pain, spasticity, or motor control, treatment of the basal ganglia in the brain for relief of Parkinsonism, loss of motor control, tremors, or intractable pain.

In one aspect, the system and method can be used to alter the function of tissue in a patient to cause the patient to experience a reduction in symptoms of a condition selected from the group comprising epilepsy, tremor, Parkinson's disease, spasticity, mood disorders, cardiac arrhythmia, depression, back pain, neurogenic pain, spinal pain, central pain, cancer pain, urinary disorders associated with the prostate and/or bladder, headache, cervical pain, and discogenic pain.

In one aspect, the system and method can include placement of an electrode applicator into or in proximity to tissue structures in a patient's body for which it is desired to alter a function. The electrode applicator can be adapted to carry or convey the signal output of a generator. The electrode applicator can be adapted to be placed into or near a functional target of a patient selected from the group of tissue comprising the spinal cord, spinal ganglia, spinal roots, peripheral nerves, thalamus, basal ganglia, pallidum, sub-thalamic nucleus, vagus nerve, epilogenic centers, the intervertebral disc, the prostate, the prostatic nerves, the bladder, and the cardiac nerves.

Forms of the modulated frequency generator and output waveforms are disclosed herein in various embodiments. Specific embodiments with temperature monitors and thermal sensing electrodes are disclosed which are suited to control the modulated system and its use.

### BRIEF DESCRIPTION OF THE DRAWINGS

In the drawings which constitute a part of the specification, embodiments exhibiting various forms and features hereof are set forth. Specifically:

FIG. 1 is a schematic diagram showing a modulated signal generator and signal applicator electrode.

FIG. 2A is a schematic diagram showing a modulated signal output from a generator.

FIG. 2B is a schematic diagram showing a constant pulse rate function.

FIG. 2C is a schematic diagram showing tissue temperatures during signal output application.

FIG. 3A is a schematic diagram showing a modulated signal output.

FIG. 3B is a schematic diagram showing a regular burst rate function.

FIG. 4A is a schematic diagram showing the durations of output waveform burst on-times versus time.

FIG. 4B is a schematic diagram showing a graph of a duty cycle of output burst on-time durations versus time.

FIG. 5A is a schematic diagram showing a distribution of on-time durations of output bursts.

FIG. 5B is a schematic diagram showing a distribution of duty cycles of output burst on-times.

FIG. 5C is a schematic diagram showing a distribution of tissue temperatures.

FIG. 5D is a schematic diagram showing a distribution of output bursts on-time durations versus time.

FIG. 6 is a schematic block diagram of the various elements of the system for generating modulated frequency signals.

FIG. 7 is a flow diagram showing a process of modulated signal output application.

FIG. 8 is a flow diagram showing a process of modulated signal output application.

FIG. 9 is a schematic diagram showing a transcutaneous surface application.

FIG. 10 is a schematic diagram showing a spinal pain relief procedure.

FIG. 11 is a schematic diagram showing a multi-electrode dorsal column application for pain relief.

FIG. 12 is a schematic diagram showing the use of an intensity-modulated frequency electrical signal applied to acupuncture needles.

FIG. 13 is a schematic diagram showing a percutaneously placed electrode and differential pulsed tissue modification zones versus thermal tissue alternation zones.

FIG. 14 is a schematic diagram showing a signal output applied to the brain.

FIG. 15 is a schematic flow chart for effects of modulated signal outputs on tissue function.

FIG. 16 is a schematic diagram showing a system for producing random on-time pulsed signals.

### DETAILED DESCRIPTION

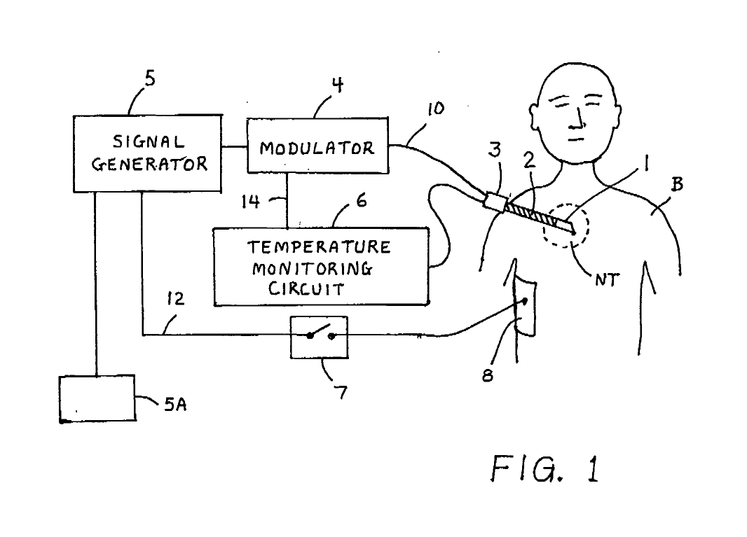

Referring to FIG. 1, an electrode with uninsulated distal conductive surface 1, for example, a conductive metal tip end, is in proximity to a region of target tissue NT (viz. illustrated schematically by the dashed boundary). The electrode has an insulated shaft 2 and connection or hub portion 3, inside of which there can be electric connections to surface 1. Connection 10 electrically connects to the surface 1 through the shaft 2 and to electronic supply units 4 and 5. Units 4 and 5 are shown outside the body, but, in other embodiments, can be miniaturized and implanted inside the body B. Unit 5 is a signal generator of signal output (viz., voltage, current, or power), and unit 4 is a modulator to modulate the amplitude of the frequency output from unit 5. The electromagnetic signal output from unit 4 and unit 5 is connected through connection 10 and hub 3 to electrode surface 1, and therefore is conductively exposed to tissue NT. As an example, unit 5 can take the form of a power source with a continuous wave oscillatory output signal such as a sinusoidally varying signal with an oscillatory frequency. In one example, unit 4 is a pulse modulation unit which switches on and off the oscillatory output signal from unit 5. Output generators or supplies and modulation circuits are known in oscillatory frequency technique (viz. Radio Engineering by Fredereck E. Terman, McGraw-Hill, New York, 1947, 3^{rd} Edition). Further shown is a temperature monitoring element or circuit 6 which connects by a cable to the electrode and to a thermal sensor (viz. thermistor or thermocouple) inside the electrode applicator or conductive tip 1 to measure the temperature of the tissue NT near the tip. Commercial examples of thermal sensing circuits and electrodes are the Model RFG-3CF rf lesion generator and associated thermal-sensing rf electrodes of Radionics, Inc., Burlington, Mass. Further, reference electrode 8 is shown in electric contact to the body B with connection wire 12 to unit 5 so as to provide a circuit for return current from surface 1 through the body B. Such reference electrodes are common with rf lesion generators as described in the above-cited reference by Cosman, et al., 1984. Element 7 is a switch or circuit breaker which illustrates that such a return current could be opened to limit such direct return current, and limit such current to inductive or reactive current characteristic of time varying circuits.

In one example, unit 4 can be a modulator that can include electronic circuitry to create a modulated envelope of the amplitude of the output of unit 5, which can be an oscillatory generator. The generator can have one or more frequency components of oscillatory wave in its output signal. The modulator can in one example turn on and turn off the signal from unit 5 to produce bursts of output signal into connection 10. The bursts can have periods of on-times during which the signal from unit 5 is sent out from unit 4 and periods of off-times during which the signal from unit 5 is significantly diminished or even reduced substantially to zero amplitude. In one example, the modulator can be adapted to produce on-times of varying and non-predetermined durations so that the bursts of output signal have unpredetermined; and/or unpredictable; and/or random lengths of time duration. Unit 4 can be adapted to produce a regular and/or periodic rate of signal output on-time bursts. The rate of bursts can be calculated as the inverse of the time elapsed between the beginning of one burst and the beginning of the next burst. In various examples, unit 4 can produce bursts of constant rate, periodically varying rates, regular repeating rates, or rates versus time that are according to a predetermined function.

The signal output from unit 4 and unit 5 are impressed upon tissue NT, which can in one example be neural tissue such as spinal nerves or roots, spinal cord, brain, etc.; or in another example tissue near neural tissue; or in another example muscular tissue or prostatic tissue. Such electromagnetic output can cause energy deposition, electric field effects, and/or electromagnetic field effects on the cells or cell substructures in the tissue NT so as to modify or destroy the function of such cells. For example, such modification of function may include reduction or elimination of pain syndromes, such as spinal facet, mechanical back pain, facial pain, and in other cases alleviation of motor disfunction, spasticity, Parkinsonism, etc., epilepsy, mood disorders, or depression. Because the output from 4 is modulated by element 5, its percent on-time is reduced so that sustained heating of tissue NT is reduced, yet the neural therapeutic effects of the impressed signal output voltages and currents on the neural tissue NT are enough to produce the pain reducing result or other clinical desired result. The unit 5 can have a power, voltage, or current output control 5A to increase or decrease the output power magnitude or modulated duty cycle to prevent excessive heating of tissue NT or to grade the level of pain interruption as needed clinically. Output control 5A may be a knob which can raise or lower the output in a smooth, verniated way, or it can be an automatic power control with feedback circuits. The temperature monitor 6 can provide the operator with the average temperature of tissue NT near electrode surface 1 to interactively access temperatures near the tip of surface 1. In one example, the operator can monitor temperature readings of a thermal sensor within surface 1. In one example, the operator can control the amplitude of the signal output during bursts of on-time so that the sensor readings are held at and/or below desired levels according to clinical needs or protocols. In one example, the temperature as sensed by the sensor in surface 1 can be held below about 45 to 50° C., which is commonly referred to as the threshold for making average heat lesions in tissue. In another example, the signal can be increased so that the sensor in tip 1 produces readings from monitor 6 that exceed in degrees centigrade (° C.): 50° C.; or 60° C.; or 70° C.; or 80° C.; or 90° C., according to clinical needs.

In another example, the signal amplitude and/or the on-time duration of output burst can be controlled by control 5A and/or unit 4 so that tissue temperatures during bursts achieve a desired level and/or do not exceed a desired level according to clinical needs. In one example, the unit 4 can have a duration control to control on-time duration, and can have a rate control to control burst repetition rates. A control in unit 4 can control non-predetermined on-time durations and/or can provide a variation range of on-time durations corresponding to a desired range magnitude, with the durations varying within that range in a random and/or unpredictable way. The variation range can be significant compared to the average duration of the on-time durations so that any particular on-time duration of a burst of the signal can differ significantly from the average duration.

In one example, the signal output from unit 5 can be a waveform comprising one or more sine-wave or sinusoidally oscillating components having one or more frequencies in the PSFR; that is, in the range of 0 to 10 KHz; or 10 to 20 KHz; or 20 to 30 KHz; or 30 to 40 KHz; or 40 to 50 KHz; or 50 to 95 KHz. The choice of frequency component range can depend on the response of the tissue and can be selected to suit clinical needs. In one example, a frequency of a frequency component in the waveform can be selected according to its expected response threshold for stimulating tissue in NT. This can have the advantage of improving the clinical result and patient comfort during the signal application to tissue NT.

In one example, the signal output from unit 5 can be a waveform comprising one or more frequencies above the PSFR. The frequencies, in one example, can be in the radiofrequency (rf) range, that is above 50,000 Hertz. Selection of frequency components above the PSFR and/or in the rf range can be made according to clinical, engineering, or tissue response objectives.

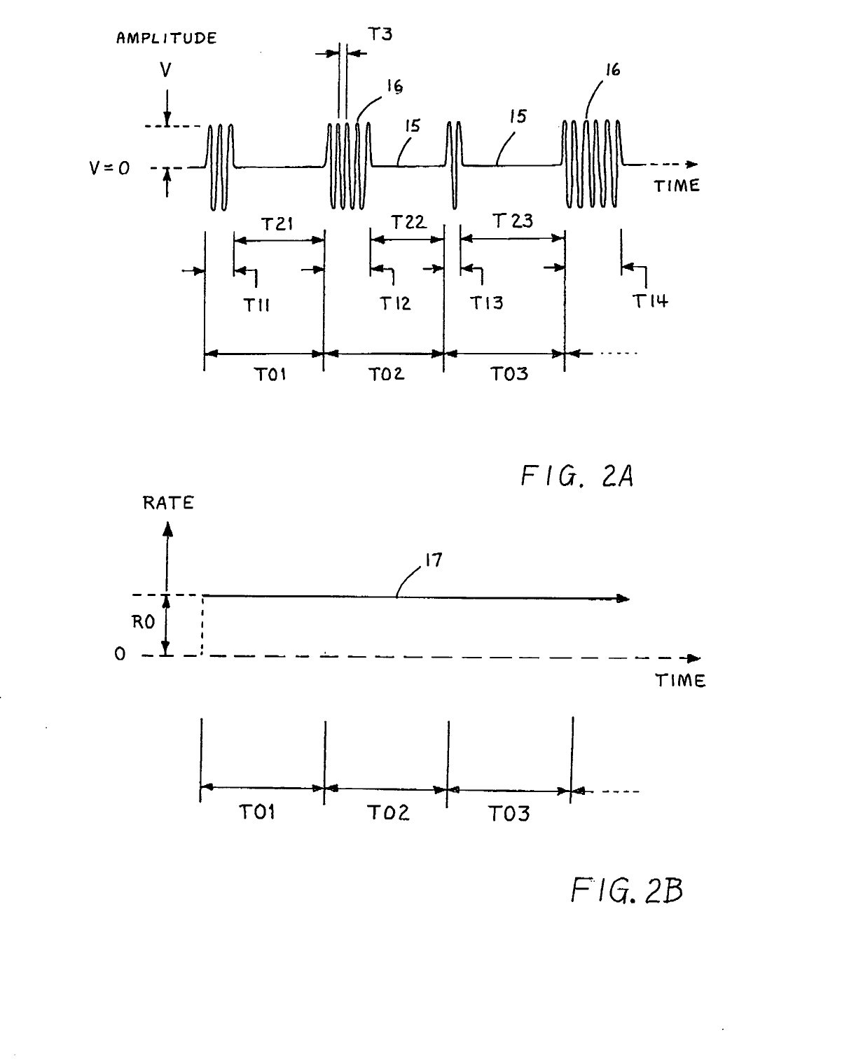

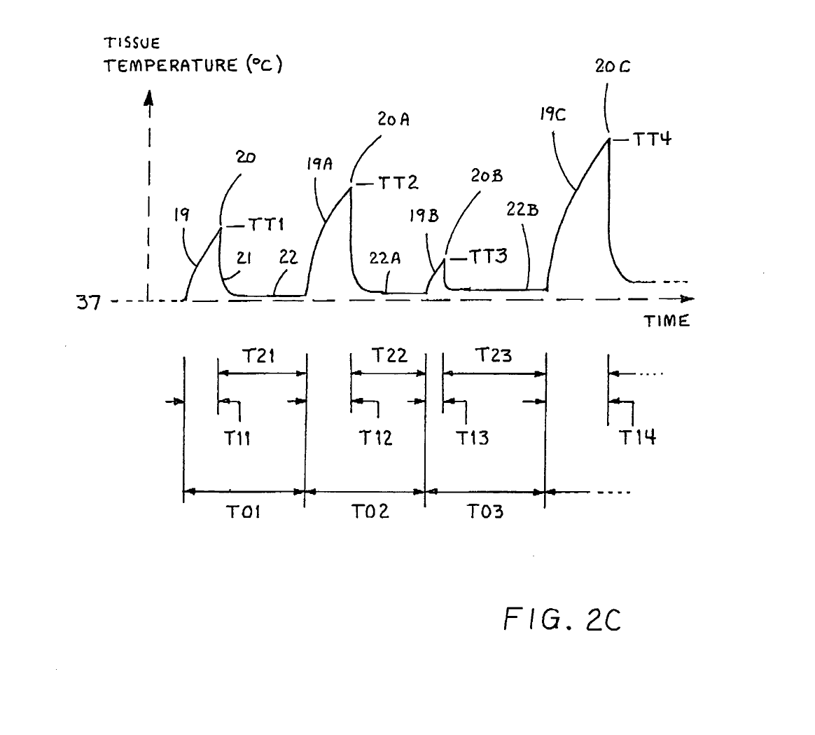

Referring to FIG. 2A, a signal output waveform is shown schematically which can be produced by unit 4 and unit 5 in FIG. 1. The output waveform has a series of bursts of an oscillatory signal, the representative bursts shown in FIG. 2A having on-time durations T11, T12, T13, T14, and the series of bursts can continue in time not shown in FIG. 2A. During these “on-times”, or bursts, the signal has an oscillatory wave or sine-wave like shape with amplitude V and time T3 between oscillatory wave peaks, such as peak 16. The oscillatory wave within each burst can be a frequency component wave with a frequency of 1/T3 for sine-waves, in units of Hertz (Hz). Between the burst on-times there are periods of lower amplitude, or “off-times”. Representative off-times in FIG. 2A have time durations T21, T22, T23, . . . etc. out to time beyond the range of FIG. 21. During the off-times, the amplitude at point 15 can be very low or even zero. In one example, the amplitude can be output signal voltage, V being the voltage maximum in on-times, and V=0 in the off-times. The series of bursts is an example of a modulated waveform. The series can go on for the time of treatment exposure of the signal to the tissue, which can be several seconds, minutes, or even hours depending on clinical objectives. The “duty-cycle” is associated with the degree of substantial on-time versus off-time.

The rate of bursts is determined by the time from the beginning of one burst to the beginning of the next burst, and these are shown for the representative bursts in FIG. 2A as times T01, T02, and T03. The rate of bursting can be defined as the inverse of the time between bursts, which in FIG. 2A is 1/T01, 1/T02, 1/T03, and that sequence can go on beyond the range of time as shown in FIG. 2A.

Referring to FIG. 2A, the on-time durations T11, T12, T22, . . . etc. out in time have: non-predetermined; or non-defined; or random values as produced by unit 4 in FIG. 1.

Referring to FIG. 2B, the pulse rate R is shown schematically versus time. In this one example, the time durations between burst T01, T02, T03 . . . etc. are shown as being equal; that is T01=T02=T03=T0 for some predetermined value of T0. In that case, the rate 17 is a constant value R0=1/T0. That is one simple example of a regular rate, and R0 can be specified in units of pulse per second (pps), often as referred to as pps (Hz).

Referring to FIG. 2C, the tissue temperature is shown schematically versus time during the sequence of signal bursts shown in FIG. 2A. During an on-time such as in period T11, the signal output V as applied to an electrode, as for example surface 1 in FIG. 1, will produce current density fields in the tissue near the tip of surface 1. The current density will produce energy deposition in the tissue causing dissipative heating, and this in turn will cause the temperature to rise as on curve 19 in the tissue during the outburst time T11. When the burst ends, the tissue has reached maximum temperature TT1 at point 20. During the off-period of low output signal, the heating ceases, and the temperature falls off along a curve 21 during time T21 according to the tissue heat conductivity, blood flow, and other physical features of the tissue region near the tip of surface 1. During the burst on-time T12, which is longer than T11, the temperature rises higher to peak value TT2 at point 20A. In the off-time T22, the temperature falls down to a level at point 22A which can be somewhat higher than the level at 22 because the average tissue temperature can rise around electrode tip. Similarly, in on-times T13 and T14, tissue temperatures rise to TT3 and TT4, respectively, over curves 19B and 19C, respectively, at points 20B and 20C, respectively. Time T23 is an off-time. On-time T11 and off-time T21 form burst T01; on-time T12 and off-time T22 form burst T02; on-time T13 and off-time T23 form burst T03. The beginning tissue temperature is shown as 37° C., which is body temperature. Depending on the signal output V of the bursts, the peak temperatures such as TT1, etc. can reach clinically significant high values which depend on the shape of the tip of surface 1, the location of the tissue relative to the tip, the level of V, the tissue electrical conductivity, the duration of the on-time, and other factors. In one example, tissue near small, or point, or sharp-edged electrode tips having applied output voltages such as 30, or 40, or 50, or 60 volts can reach peak tissue temperatures in ° C. of: 40; or 50; or 60; or 70; or 80; or 90; or 100; or more. In some cases, the tissue temperature can have peaks that substantially exceed the commonly referred to lethal tissue temperature range of 45 to 50° C.

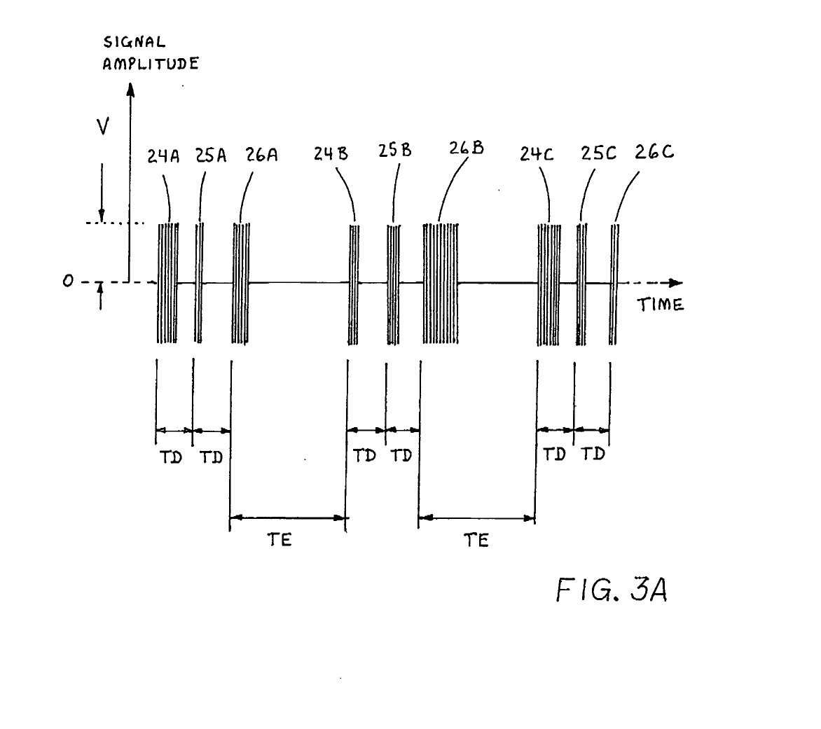

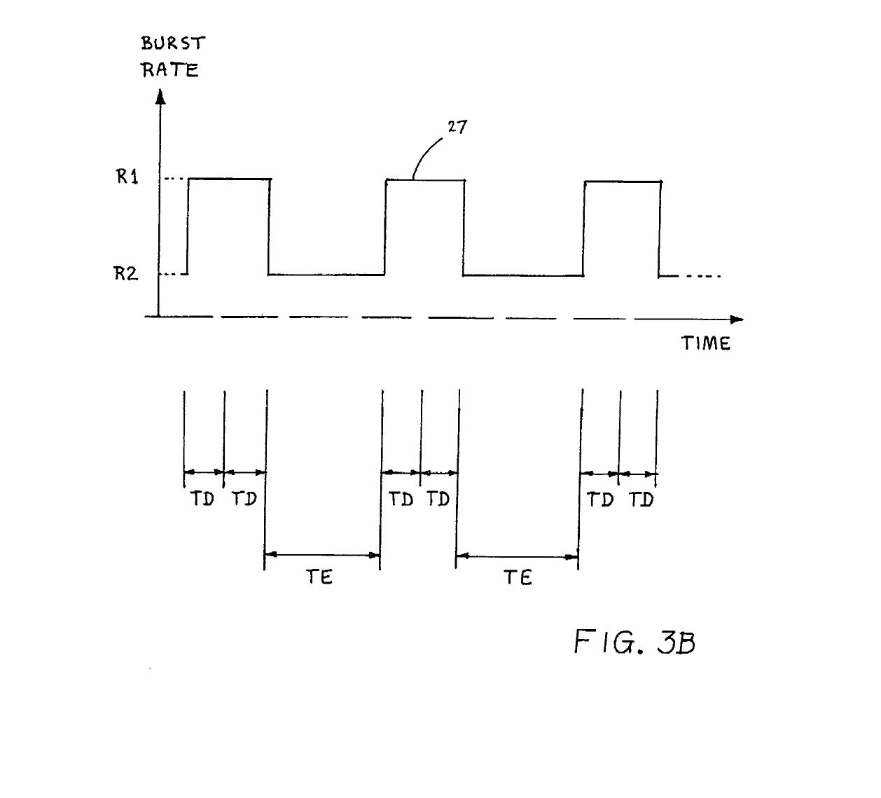

Referring to FIGS. 3A and 3B, a signal output waveform is shown schematically with random pulse widths, and regular pulse rates that are not constant. In FIG. 3A, the bursts of output come in groups: 24A, 25A, and 26A; and 24B, 25B, and 26B; and 24C, 25C, and 26C; etc. in time. Within each group the bursts are spaced by duration TD. The last burst of each group, like 26A, is spaced from the first burst of the next group, like 24B, by time TE. The rate R2 within the group is 1/TD and the rate R1 between the groups is 1/TE. FIG. 3B shows the rate change 27 versus time and shows the change in rates from R1 to R2 during times TD and TE. The rate change is regular, and in this example, periodic. The on-time for each burst, for example the width of the shaded areas corresponding to bursts 24A, 25A, 26A, 24B, . . . etc., is changing, and the burst widths are: non-predictable; and/or random; and/or non-definable.

In other examples, the burst rate R can vary periodically and/or according to a known function of time. An advantage is that the average tissue modifying effects or the average temperature elevations of tissue can be controlled smoothly to suit clinical needs.

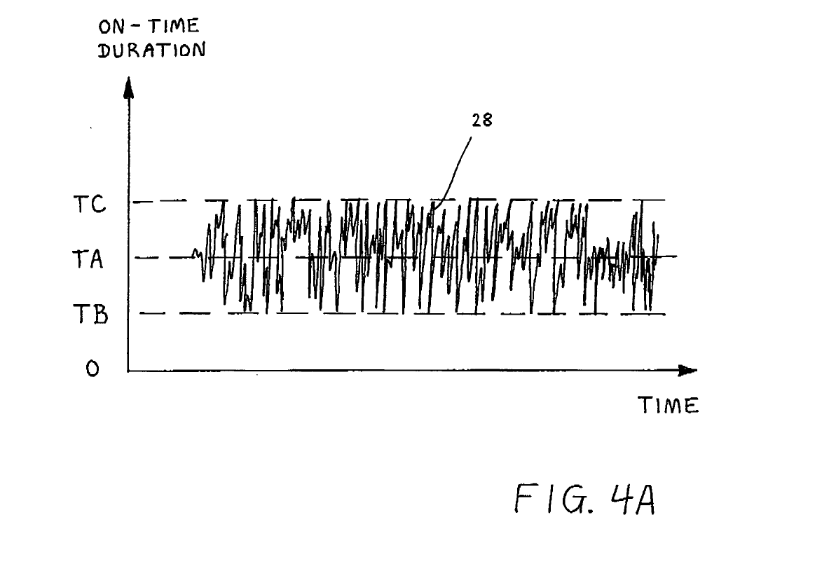

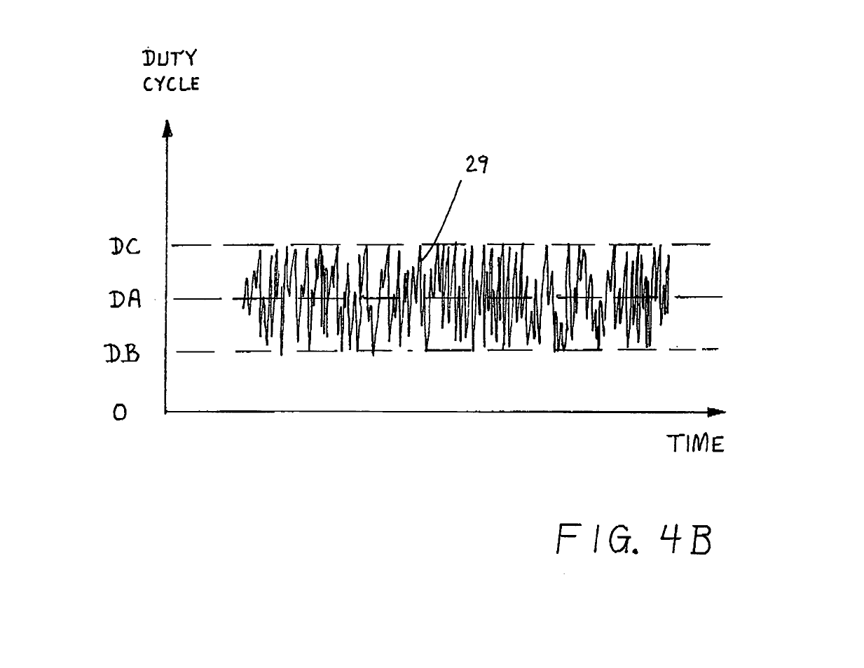

Referring to FIG. 4A, the graph 28 of on-time durations of signal output bursts, in one example, are shown schematically over a period of time of treatment. In one example, the durations are non-predetermined and random in the values and range between a maximum of TC and a minimum of TB. The distribution of durations, in one example, has an average of TA. In FIG. 4A, the horizontal time axis spans a wide time range so that the random character of the burst durations can be schematically seen over a large number of bursts. In one example, there is no correlation between the on-time duration of one burst with duration of the next burst and no correlation between duration of any two or more bursts that are selected.

In other examples, the range of possible on-time durations of burst can be unlimited.

Referring to FIG. 4B, the duty cycle 29 of the signal waveform is shown schematically versus time for a waveform with randomly varying on-time durations and with a constant repetition rate of bursts. The duty cycle can be calculated as the ratio of the on-time duration of a burst divided by the time between the beginning of the burst and the beginning of the next burst. It is common that the duty cycle can be expressed as a decimal or as a percentage. In one example, the duty cycle has non-predetermined and/or random variations between a maximum of DC and a minimum DB with an average and/or mean value of DA. In one example, the variation of the duty cycle between DC and DA significantly exceeds the noisy variations of a duty cycle which occur for a predetermined duty cycle caused by ordinary noise in electronic circuits. For example (DC−DA)/DA in one example can be greater than: 0.05; 0.1; 0.2; 0.5; or more depending on the clinical needs. To give one example, if this ratio is pre-selected to be 0.5 through circuit designs or controls in modulator element 4, then any given duty cycle value can be 50% more or 50% less than the average value DA and therefore cannot be given as approximately equal to DA.

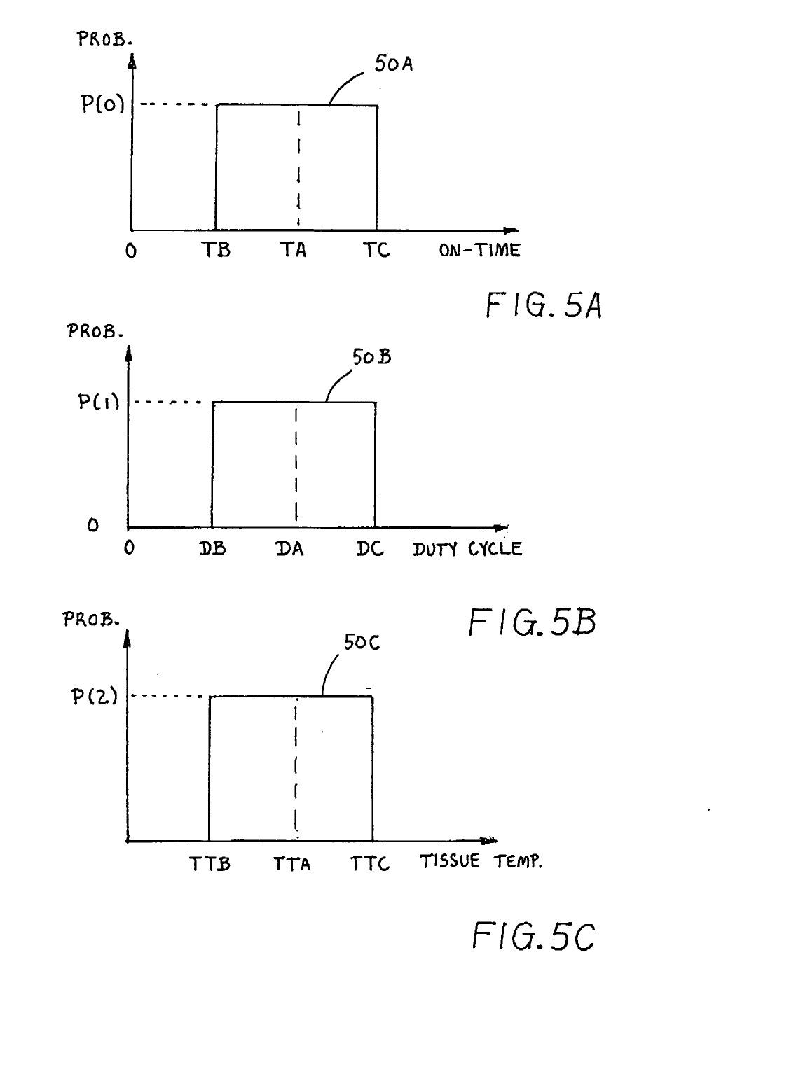

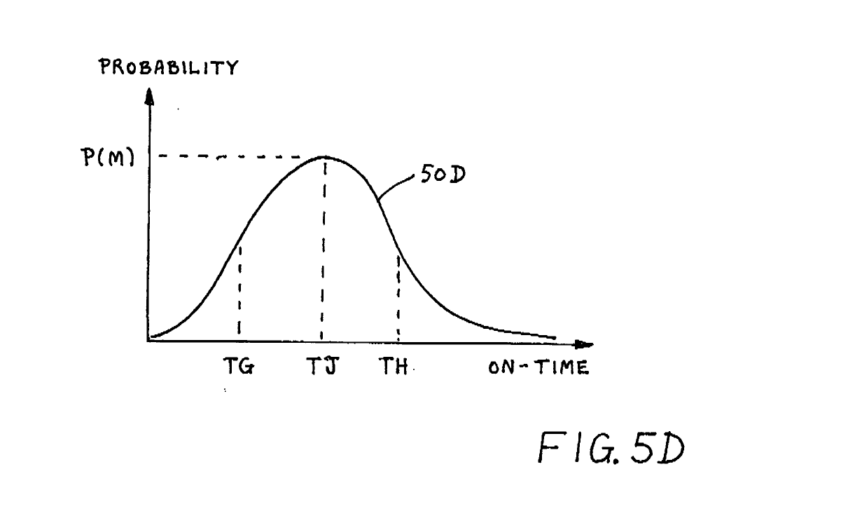

Referring to FIG. 5A, the probability distribution 50A of waveform burst on-time duration values are shown in one example. The values range from a minimum TB to maximum TC, and the probability of any value between TB and TC is constant at P(0). On-times can occur in a non-predetermined; or random; or non-predictable way within those distribution limits. In one example, an average on-time of TA can be pre-selected or predetermined. In another example, an average on-time is not pre-selected.

Referring to FIG. 5B, the probability distribution 50B of duty cycles of output bursts are shown in one example. The values range between DB and DC with an average value of DA. Duty cycle values occur with equal likelihood P(1) within that range.

Referring to FIG. 5C, the probability distribution 50C of peak values of tissue temperatures for a portion of tissue near the output applicator electrode is shown in one example. They range from TTB to TTC and have an average value of TTA. For random pulse on-times, the peak tissue temperatures are distributed randomly in this range. The range TTB to TTC depends on signal output amplitude, electrode geometry, on-time duration, tissue sample volume and location with respect to the electrode, and tissue electric conductivity and heat capacity.

Referring to FIG. 5D, a probability distribution graph 50D of on-time durations is shown schematically in one example. The values of on-time durations can range from near zero to large values, and the probability versus value curve 50D varies smoothly with a maximum P(M) at on-time value TJ. In one example, TJ can be the average on-time value, and it can be predetermined and/or set by the operator by modulation on unit 4 of FIG. 1. The values TG and TH can represent half-values of on-times for which the probability function drops to one half of P(M). The on-time values can be non-predetermined; or random; or uncorrelated. Any particular on-time duration corresponding to a particular signal burst can differ widely from the average value or maximum value TJ.

In one example, the signal output voltage can oscillate with frequencies 1/T3 between the voltages of +V and −V during the on-times T1. Accordingly, an electric field is produced around the region of the electrode applicator, as for example the exposed electrode tip 1 in FIG. 1. The electric field has a modifying, or pain-relieving, or neural-altering effect on the tissue near or among the nerve cells and fibers. Pain relief and neural modification can accordingly be accomplished by this bursting voltage waveform and accompanying electromagnetic field and accompanying current field among the neural and tissue cells. During the off-time period there can be minimal or no voltage (i.e., V=0 at the electrode applicator), and thus no electric field or electric currents in and among the neural tissue. During that off-time period, much less and/or no heat deposition in the tissue is produced. Over the entire cycle, from on-time period through off-time period, the average and peak values of energy deposition, electric fields, and tissue temperatures can be adjusted, for example, by selection of: amplitude V; and/or on-time; and/or off-time.

In one example of values for parameters in an interrupted oscillatory frequency waveform as in FIG. 2A, the overall pattern of the repeating waveform has a total period of one second, meaning that the T01=T02=T03= . . . =1 second. The on-times T11, T12, T13, . . . can have an average value of 20 milliseconds. The frequency of the sinusoidal frequency component 1/T3 in the on-time bursts, which can be called the carrier frequency, can be in the range: 0 to 1 KHz, if very low frequency stimulative responses are indicated; or 1 to 5 KHz if low frequency stimulative responses are indicated; or 5 to 10 KHz, if somewhat higher frequency stimulative responses are indicated; or 10 to 20 KHz, if intermediate frequency stimulative responses are indicated; or 20 to 40 KHz, if intermediate-to-high frequency stimulative responses are indicated; or 40 to 60 KHz, if high frequency stimulative responses are indicated; or 60 to 90 KHz if very high stimulative thresholds or responses are indicated; or greater than 90 KHz; or within the rf range of greater than 50 KHz, according to clinical needs and desired effects on tissue.

The voltage V in FIG. 2A or 3A can be in the range of: 0 to 10 volts; or 10 to 20 volts; or 20 to 30 volts; or 30 to 40 volts; or 40 to 50 volts; or 50 to 60 volts; or higher depending on the threshold stimulation response indicated, the variation in tissue temperatures desired, or the time-course of output exposure desired. The chosen amplitude V can be chosen to give a desired level of neural modification or of pain relief.

In one example, the output waveform in FIG. 2A can be used to relieve pain. The temperature of the tissue NT near or at an electrode tip such as surface 1 in FIG. 1 can be controlled by the waveform or waveform parameters such as by the amplitude level such as V in FIG. 2A and the on-time durations and burst rate. For a predetermined value of the average value of the on-time durations and the burst rate, the level of V can be varied to achieve a desired average tip temperature at the electrode applicator. Depending on the clinical indications and need, the average temperature as sensed by the thermal sensor in the electrode tip of surface 1 can be controlled to be in the range of: 37° C. to 40° C., if desired to be well below lethal levels; or 42° C. to 45° C., if desired to be in a typically reversible temperature zone; 40 to 42° C., if desired to be in a non-lethal to reversible temperature zone; or 45 to 50° C. for borderline lethal temperatures; or 50 to 60° C. to destroy a small zone around the electrode; or 60° C. to 100° C., if larger lesion sizes are desirable. The average tissue temperature and the time of output exposure of the waveform of FIG. 2A can affect the amount and zone of neural tissue modification at non-lethal temperature levels and the amount and zone of tissue destruction at lethal temperature levels.

As described in connection with FIG. 2C, temperature bursts or spikes in specific tissue volumes near an electrode applicator tip such as tip 1 in FIG. 1 can exceed the average tissue temperature near the electrode. Peak flash temperatures during the on-time can stun, or change, or modify, or kill tissue cells, and these effects can depend on peak burst temperatures and the duration of the burst in the on-time.

In one example, waveform parameters including: the signal amplitude; the average on-time duration; the distribution of on-time durations of the bursts; the rate of output bursts; and the combination of carrier wave oscillation frequencies can be chosen or selected in element 4 and 5 in FIG. 1 to achieve a desired tissue modification effect.

In one example, waveform parameters can be chosen to achieve desired levels of flash temperatures and average temperatures to achieve a desired neural tissue or other tissue modification. In one example, flash temperature above the so-called lethal level of 45 to 50° C. can stun or modify the neural or other tissue, but not totally kill the tissue. Parameters can be chosen to give bursting flash temperatures of: 50 to 60° C.; or 60 to 70 C; or 70 to 80° C.; or 80 to 90° C.; or 90 to 100° C.; or even higher depending on clinical needs and desired effect.

Referring to FIGS. 2A through 5D, the choice of on-time average duration, range of random on-time burst durations, burst rate in pps (Hz), and/or amplitude V can be directed at achieving a desired clinical effect. In one example, using an electrode tip of diameter of about 1 mm or less with pointed or sharp contours, amplitudes of V of 30 to 100 volts; average on-time duration of 10 to 20 milliseconds; and burst rates of 1 to 10 pps (Hz), peak burst temperatures in tissue locations near the sharp portions of the electrode tip can exceed 50° C., 60° C., 70° C., 80° C., or higher depending on a given burst's on-time duration. Average temperature measured by a sensor in the electrode tip during this signal application can read below 45° C. to 50° C. This combination of parameters can have a desired effect on tissue near the electrode.

In another example, it can be desirable to reduce the peak burst temperatures during on-times by reducing the average on-times to be in the range of: 0.1; or 0.5; or 1; or 5 milliseconds. The rate can be increased to maintain a desired overall exposure of the tissue to the signal output effect. In one example, the product of average on-time duration times the number of bursts per second can be maintained by choosing shorter on-times and longer burst rates to maintain burst peak temperatures to a desired clinical level.

In one example, the spread of non-predetermined burst on-times can be chosen to spread or average the effect of signal output effects on tissue. For example, an average on-time of 10 milliseconds can be chosen and a distribution of random burst on-times between 5 and 15 milliseconds can be chosen to smear out the burst effect on the tissue for desired clinical effect. In another example, shorter or longer average on-times can be chosen and random on-times of say 50% above and 50% below can spread the clinical effect of the output according to desired tissue modifying needs.

Referring to FIG. 2A, in one embodiment, signal output on-times can be held constant, burst rate can be held constant, and the amplitude V can be varied in a non-predetermined or random way around a chosen average amplitude VAVG. This can spread the signal effects on tissue over randomly varying magnitudes to achieve a desired clinical effect. In one example, percent random variation about VAVG can be in the range: 1 to 10; or 10 to 20; or 20 to 50; or more. Such a waveform can be generated by random noise generators, spark gap signals, or other noisy signals that are known in the field of signal generation (viz. see the texts by Terman and by Schwartz cited above). Filtering can be applied in the wave generator and power amplifier so that only frequencies in the physiologic stimulation range can be present in the waveform.

The waveform on-times, rates, off-times, and carrier frequencies can have various values depending on clinical needs. For example, the average on-time can be 100 microseconds or less. Average on-times can be in the range of: 100 to 1000 microseconds; or 1 to 100 milliseconds; or 100 to 500 milliseconds; or longer. Off-time durations can be substantially longer than on-time durations. For example, the ratio of off-times to on-times can be in the range of: 1 to 10; or 10 to 100; or 100 to 1000, depending on desired clinical effect on target tissue. Shorter on-times can be used with larger V amplitudes to produce similar average temperature elevations in the tissue. Larger V volumes can produce higher flash temperatures in the tissue and higher electromagnetic field effects. Shorter on-times for comparable amplitudes V can produce reduced peak temperature bursts while maintaining the strength of fields and field-effects in target tissues. Tissue constituents can respond differently to different carrier frequencies 1/T3. The choice of this signal output parameter can depend on the thermal and the field response of tissue to produce a desired tissue modification.

Returning to FIG. 2A, in one example, the carrier frequency 1/T3 can vary in a predetermined manner. In another example, the carrier frequency can vary in a predetermined or regular manner.

In one aspect, the use of non-predetermined or of varying and non-periodic parameters can have the advantage of smoothing the tissue response to the applied signal over a wider parameter range to broaden the response and threshold of tissue to the applied signal.

In other examples of the waveforms corresponding to FIGS. 2A to 5D, different waveform parameters can be used. In one example, a slowly varying baseline of non-zero value can be used. The time average of the signal can be non-zero. The carrier frequency 1/T3 can be non-constant. Varying, or combined, or superposed carrier frequency waveforms can be used and the combined or composite carrier frequency waveforms can be interrupted or modulated. Pulse waveforms that modulate carrier frequencies can be shaped in a variety of ways, for example, with fast rising leading edges and slow or falling off or exponential trailing edges. The signal generator waveform can have a peak intensity to yield a high peak electromagnetic field or current density in the target tissue while maintaining the average power deposition in the tissue at a sufficiently low level to prevent heating above average lethal tissue temperatures (viz. 40 to 50° C.).

If the waveforms have one carrier frequency component with for example frequency 1/T3, then the overall frequency spectrum, derived from the Fourier transform, of the modulated carrier component will typically have dominant frequency components centered around the corner frequency 1/T3.

In another example, the waveforms can be produced having several carrier frequency components with different frequencies. The frequencies can be chosen to be in a narrow range to cover a broad portion of the PSFR. Different carrier frequencies in the PSFR can produce different stimulative responses and level thresholds depending on nerve type, tissue location, and location of the electrode to the nerves and surrounding tissue. In one example, it can be advantageous to have only one carrier frequency in the PSFR. In another example it can be advantageous to have several carrier frequencies in the PSFR. In another example, the waveform can have carrier frequencies in the PSFR admixed with carrier frequencies above the PSFR. In another example, the carrier frequencies are above the PSFR.

In another example, the waveforms can have varying amplitudes V during the burst on-times T1. During one on-time the voltage can be V1, during the next on-time the voltage can be V2, during the next on-time the voltage can be V3, and so on in a sequence. In one example, the pattern of voltage variation can be a repeating or a regular pattern. In another example, the pattern can be a non-repeating variation. This can have the advantage that the bursts with high amplitude can be used to probe stimulative response levels and other bursts of lower amplitude can produce a neural modification effect with lesser stimulative response. The threshold for stimulative response depends on signal amplitude (such as voltage or current amplitude of the burst signal) and on the frequency of the sine wave component frequency. Some fraction of the bursts can be at lower stimulation response threshold and some fraction of the bursts can be at a higher stimulation threshold so as to achieve a desired balance of patient sensation and neural modification effect from the signal as a whole.

In one example, the waveform parameters can be chosen so that the average temperature and the spiking temperatures during the on-burst times in the tissue near the electrode are maintained so that at least a portion of the tissue in the target volume is not killed. In one example, the average temperature near the electrode can be kept less than about 45 to 50° C. This can have the advantage that at least a portion of the target tissue will not be killed during the tissue modification process.

In another example, the waveform parameters can be chosen so that the average temperature in the tissue near the electrode equals or exceeds the lethal level of about 45 to 50° C. This case can have the advantage that some zone of tissue will be killed while another zone of tissue farther from the electrode will be modified by the signal fields.

In the examples of waveform parameters being chosen to affect average temperatures, the average temperature can be defined as or determined by the time averaging of temperatures in tissue near the electrode applicator or can be the temperature as measured by a temperature sensor(s) in the electrode or near the electrode applicator. In one example, average temperature can be the time-average of temperatures in a selected portion of tissue over the time span of one on-time period plus the following or previous off-time period. In another example, the average temperature can be the time average of temperatures in selected tissue averaged over many burst cycles of on-time periods and off-time periods within the overall treatment time. In another example, the average temperature can be a running time average corresponding to a prescribed averaging time interval. In another example, the average temperature can mean the time-average over the entire treatment time of temperatures as measured by a temperature sensor in the output electrode applicator and/or a separate thermal sensor which has intrinsic averaging caused by the thermal response of the sensor and applicator characteristics and/or by the averaging of the thermal and electrical detection and readout response times.

In another example, the waveform can comprise a mixture of modulated carrier frequencies within the PSFR and modulated carrier frequencies above the PSFR. The modulated PSFR components can test stimulative response as well as modify nerve function, and the modulated components above the PSFR can produce only neural modification effects. This can have the advantage of modulating the stimulative response as the signal exposure progresses.

In one example, electrode tips, such as surface 1 in FIG. 1, can have diameters of: 0.1 to 0.5 mm for small targets; 0.5 to 1.0 mm for intermediate size targets; or 1 to 2 mm for large targets; or larger than 2 mm for very large targets. Exposed tip lengths of the electrode tip such as surface 1 in FIG. 1 can also range from: 1 to 10 mm; 10 to 20 mm; or 20 mm or more and can be selected to suit target size.

The electrode applicator can be inserted into or placed near targeted neural structures such as the brain or peripheral nerves or peripheral nerve ganglia to accomplish pain relief or other neurological alteration. Variation of signal output parameters can be made and various geometries of conductive electrode or applicator can be chosen to suit desired clinical effect or specific anatomical target region. Illustrations of a wide variety of such electrodes are illustrated by the product line of Radionics, Inc., Burlington, Mass. Pointed or sharpened electrodes, such as illustrated schematically by electrode tip 1 in FIG. 1, are useful for penetration of the electrode through the skin to the target neural tissue site, and electric or current fields of higher intensity will be present at a sharpened point for a given applied voltage, such as V in FIG. 2A, which can be effective in altering neural function.

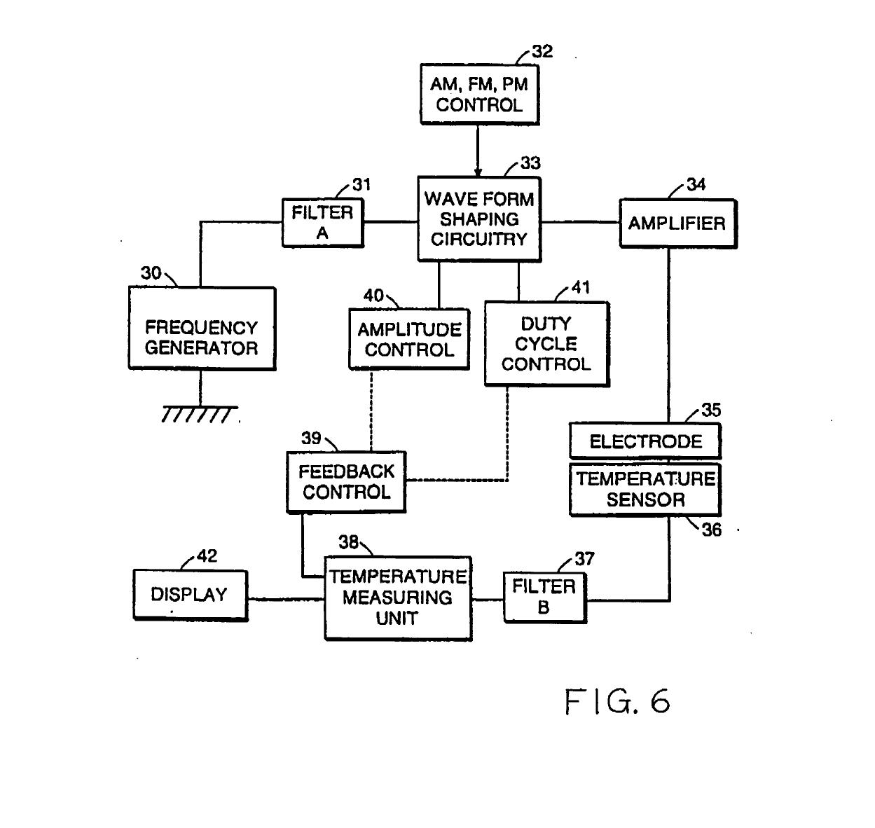

Referring to FIG. 6, a block diagram represents one embodiment of a system to generate a waveform for signal output generation such as referred to by unit 4 and unit 5 in FIG. 1. Element 30 represents a generator of a signal output which can have one or more sine-wave frequency components in the PSFR. The signal output from element 30 goes into filter 31 which assures that only the desired frequency components are filtered out. The signal is then fed into element 33, which is a waveform shaping circuit, and will shape the waveform input from element 32, which provides amplified modulation and/or frequency modulation and/or phase modulation control. Circuits of this type can be found, for instance, in Radio Engineering by Terman (cited above). Additional waveform shaping can be done by element 40 and element 41, which can control the amplitude of waveform and/or the duty cycle of the waveform, respectively. This resultant signal is then fed into a power amplifier represented by element 34. This is a wide band amplifier used to increase the signal to power levels appropriate for clinical use. This energy is then delivered to the patient via an electrode depicted as element 35. Element 30 can generate signals according to the examples of waveforms as illustrated in the other figures and accompanying description contained herein.

Element 33 can include circuits to produce on-time bursts of different durations. The durations can be made: non-predetermined; or random; or non-predictable with a variation range around an average on-time that is chosen; predetermined; and/or selectable by the operator.

A temperature sensor or plurality of temperature sensors, represented by element 36, can also be placed and connected in proximity to this electrode so as to insure that the temperature does not exceed desired limits. This temperature sensor signal is fed through element 37, which is a special filter module used to eliminate unwanted frequency components, and thus not to contaminate the low-level temperature signals.

The temperature signal is fed to element 38, which is a standard temperature measuring unit that converts the temperature signal into a signal that can be used to display temperature and/or to control, in a feedback manner, either the amplitude and/or the duty cycle of the waveform. In this way, power delivery can be regulated to maintain a given set temperature or remain below a given set temperature. This flow is represented by element 39, which is simply a feedback control device. The dotted lines from element 39 to element 40 and element 41 represent a feedback connection that could either be electronic and/or mechanical. It could also simply be a person operating these controls manually, based on the visual display of temperature, as for example on a meter or graphic display readout 42.

In FIG. 6, the filter 31 and filter 37 can be designed to properly pass the desired frequency or frequencies in the PSFR that will be modulated by element 33. For example, a carrier frequency with 10 KHz will require a band-pass or an active filter 31 to efficiently pass 10 KHz signals but inhibit signals substantially lower than or higher than 10 KHz. A filter band window, in one case, of 2 KHz around 10 KHz center can be adequate.

Referring to FIG. 7, the operation of the system and method is shown with a flow diagram. Assume that an electrode, such as for example surface 1 in FIG. 1, is placed in contact with the patient's body, or inserted into the patient's body and connected to a modulated frequency generator (represented by unit 5 and unit 4 in FIG. 1) in the manner described above. Once the electrode is in place, a clinician can decide on the desired electrode parameters and modulated waveform parameters that should be used. This is indicated by initialization block 100 in FIG. 7. For example, for a given electrode geometry or location of surface 1 in the patient's body, it can be decided that a certain average duty cycle of frequency signal, voltage, current, or power level of frequency carrier or a mixture of frequency components in the PSFR; or in the rf range; or above the PSFR is desirable. In one example, to achieve a desired stimulative response during application of signal, a particular frequency can be more effective to produce this at certain voltage thresholds; see for example the paper by W. W. Alberts, et al. cited above which shows response levels versus carrier frequency. In another example, the modified PSFR frequency generator can have fixed parameters, which are used universally for certain types of procedures, in which case the initialization block element 100 in FIG. 7 is not present. This is symbolized by the dashed line between block element 100 and block element 102. In block 100, the choice of average burst on-time can be made and/or the range of variation of the random burst on-time can be made.

Suitable electrode geometry, for example, sharpened electrode shaft, catheter-type electrode, surface electrodes for skin application, flattened electrodes for cortical or spinal cord application can be made (block 100).

Block 102 indicates the start of the signal output application in which an “on” button may be pushed and the elevation of signal voltage, current, or power (level) is started. In a case where the temperature sensor is disposed in or near the electrode applicator connected to the patient's body, the temperature monitor 1031 is indicated, which can sense that temperature and monitor or read it out to the clinician. Alternatively, temperature sensing can be made at a position away from the output applicator. For instance, a separate temperature sensor can be inserted at a position located at a distance from the applicator electrode. Increasing the output level 102 to achieve the neural modification effect (for example, pain relief for the patient) is accomplished by the electromagnetic, electric, or other aspects of the applied field in the presence of the neural structures. In one example, if the temperature monitor 1031 shows that the temperature of the tissue is being elevated, then the decision block 104 determines that if these levels are reached, a reduction of the applied power (block 105) can be implemented so as to reduce the temperature monitored level in block 1031. If lethal temperature levels have not been reached, there is the option to continue with raising the output level or to hold it static at a desired, predetermined level until the proper clinical effect has been reached. In another example, step 104 can involve deciding what temperature above the range 40° C. to 50° C. is an acceptable limit to achieve by signal increase. At end point of a particular rf level or time duration for the exposure indicated by element 106 may be utilized, and when a rf level or time has been reached, then the unit may be shut off, as indicated by block or element 107.

Block 1032 indicates the step of monitoring the stimulation response of the patient to the signal application. The signal has modulated frequencies in the PSFR, and monitoring the patient response to the signal can indicate the appropriate signal level and if the desired clinical result and neural modification level has been reached. It is an advantage of the method and system that simultaneous monitoring of clinical effect can be done as the signal application therapy is proceeding. Block 104 determines if the desired level is reached based on block 1032 and signal reduction (105) or decision on level and time exposure (106) can determine if the exposure should end (step 107).

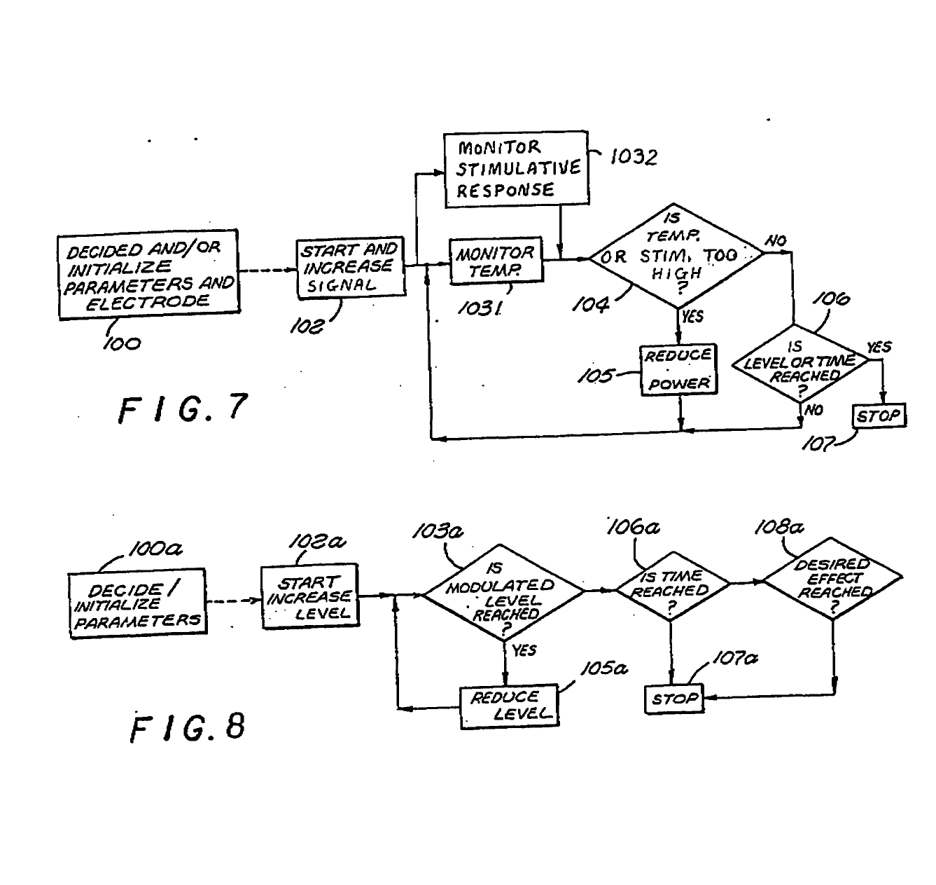

Referring to FIG. 8, another flow diagram for cases is shown where temperature monitoring is not conducted. In such situations, it can be decided by block element 100a that some target parameters for the output signal, such as voltage, current, frequency of carrier and waveform for the applied signal, or power level, will be used in a given anatomical region and for a given electrode. The signal level is increased in step 102a, and if the level of modulated frequency output is reached (determined by decision block or element 103a), then, a feedback may take place to reduce that level as represented by block 105a. Element 103a can simply be a manual control or output control knob or it can be done by electronic feedback on the power amplifier or signal generator. If the parameter criteria for an adequate procedure is a certain time duration, then in the decision process, if that time is reached, step 106a may be actuated and the system stopped when that desired time duration has been reached. Variations of pulsed radiofrequency signals could be applied ranging from several seconds to several minutes or more depending on the clinical conditions. In other examples, time duration is not the desired end point parameter, then possibly the observation of a desired clinical effect such as abolition of pain, tremor, spasticity, or other physiologic parameter may be the desired criteria, as shown by element 108a, again to make the decision to stop the procedure, as in element 107a. Using the modulated signal having components in the physiologic stimulation frequency range, the desired effect can be the change in the stimulative response level or threshold (step 108a). When this effect is reached, then the procedure can be stopped (step 107a).

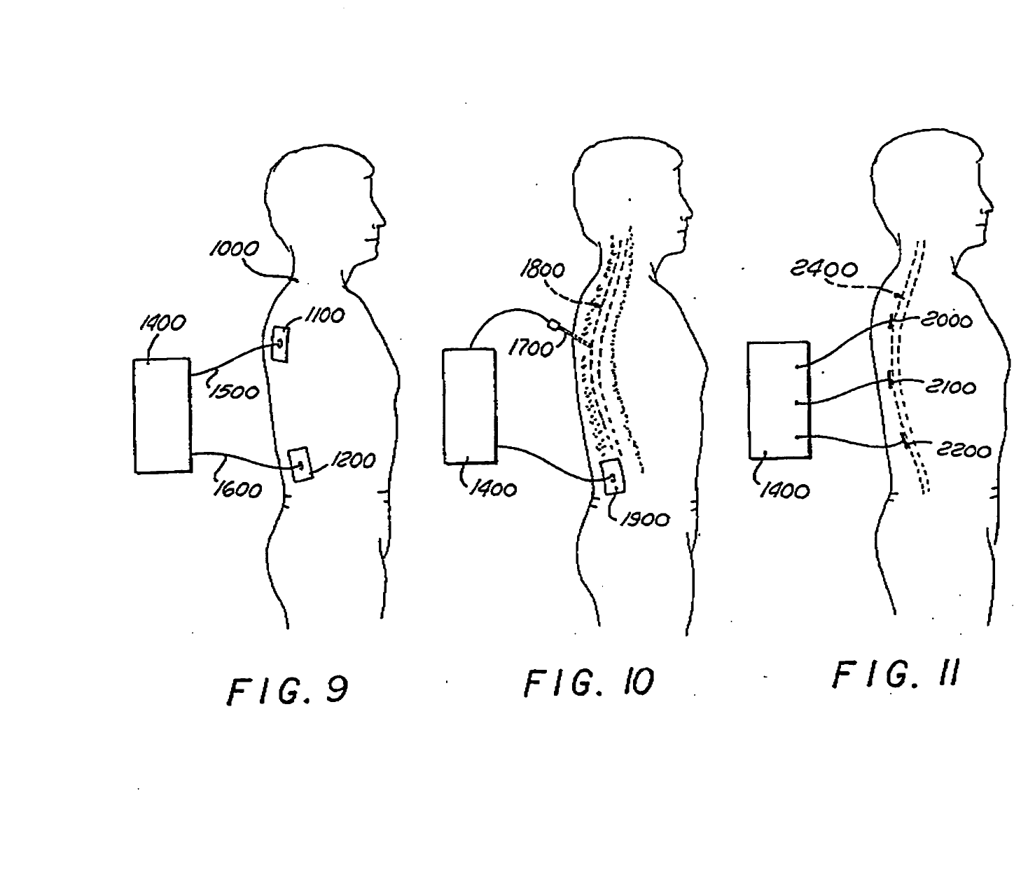

Referring to FIG. 9, the patient's body 1000 can have applied to its surface electrode 1100 and surface electrode 1200, which may be connected to the signal generator 1400. Generator 1400 has a modulated frequency signal such as described above. Its output can be applied via wire 1500 and wire 1600 to the surface-based applicators to induce neural modification in nerve cells at the surface of the body or just below the surface, or in substantial tissue depths depending on clinical needs. Electrodes such as electrode 1100 or electrode 1200 can be positioned over nerve trigger points, spinal nerves, neural dermatomes, the vagas nerve, the sciatic nerve, nerves in the limbs, and other nerves to treat pain, depression, motor disfunction, or other neural or motor syndromes.

Referring to FIG. 10, an electrode shaft 1700 is inserted near or into the patient's spinal column 1800 and associated neural structures. This is done in the case of facet denervation, dorsal root ganglion modification, spinal cord structures, or other neural structure modification in or near the spine. The generator 1400 is similar to one described in FIGS. 1 and 6 above with a modulated frequency signal to cause neural modification of the spinal nerves in and around the spinal column 1800. This can be effective in alleviating back pain, headache pain, motor disfunctions, or other spinal diseases. The reference electrode 1900 is applied to the body as a return current source.

Referring to FIG. 11, a spinal cord or dorsal column application is shown in which multiple electrodes 2000, 2100, and 2200 are applied to or near the spinal column or spinal cord 2400. Electrodes of this type can be catheter-based or flat-strip type electrodes, some examples of which are commercially available for dorsal or spinal stimulation from Medtronic, Inc., Minneapolis, Minn. The modulated signal generator 1400 is shown with multiple outputs connected to electrodes 2000, 2100, and 2200, which may be implanted or on the surface of the spinal cord, as illustrated by element 2400. The electrodes 2000, 2100, or 2200 may be greater in number, and they may be inserted through a catheter or serial string element, which may be tunneled near the spinal cord percutaneously. Application of the neural-generated output from 1400 may cause pain relief, relief of spasticity, relief of other muscular, motor, or neural disfunctions by the neural modification as described in the above embodiments. In another example, generator 1400 can be miniaturized and battery or induction powered to be fully implanted beneath the patient's skin. Examples of fully implanted generators are given by stimulators made by Medtronic, Inc.

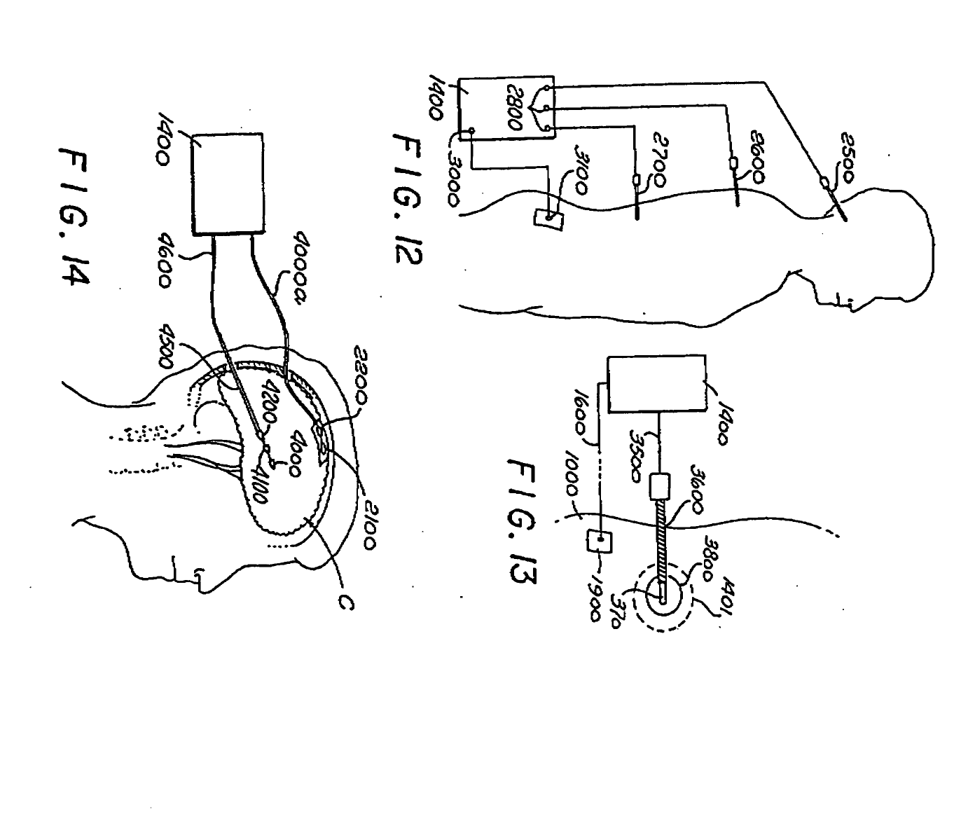

FIG. 12 shows another embodiment of the present invention in which multiple electrodes 2500, 2600, and 2700 are inserted into various portions of the body and connected to a signal generator or modulated carrier frequency generator 14 via the outputs 2800, which can be coincident or sequenced. Connection 3000 is made via connector wire to electrode 3100. Electrode 3100 can be a reference electrode. Electrode 3000 can also be used as an area electrode for application of the signal output to nerves. The percutaneous electrodes 2500, 2600, and 2700 can be electrodes that comprise a metal shaft or wire shaft, and they can be of fine gauge such as for example acupuncture-type electrodes. Acupuncture electrodes or other electrodes can be put into various trigger zones within the body, and the modulated frequency signal from generator 1400 can enhance the anesthetic or pain relieving effect of these electrodes. Thus, the present system can be used to enhance or augment acupuncture type techniques.

FIG. 13 illustrates the differential effects of the modulated waveform stimulation frequency range fields for tissue or neural tissue modification. Electrode 3600 with insulated shaft, except for exposed tip 370, is inserted into the body or into an internal organ. The tissue of the body is element 1000. The electrode is connected via connection 3500 to a high frequency generator 1400, which can have a reference line 1600 connected to reference electrode 1900. The dashed portion of line 1600 illustrates that this connection can be or cannot be made by an electric current-carrying wire, but it rather can be a reactive or capacitive connection with no wire. The generator can produce sufficient root means square (RMS) power output to produce an isotherm contour 38, corresponding to a temperature greater than the defined average lesion temperature of approximately 45° Celsius. For example, the line 3800 can represent an isothermic surface of 50, 55, or 60, or more degrees, and the tissue within the volume can be killed by a conventional heat lesion. Nonetheless, the electric fields and current generated around the electrode tip 3700 from, for example, an electric voltage output from pulse modulation generator 1400 can produce electric fields that can modify neural tissue out to a larger surface, illustrated by the dashed line 1401. Thus, the tissue between surface 3800 and surface 1400 can be, for example, neural tissue that is modified by peak voltage or current intensities from the modulated electronic output of generator 1400. That output, for example, can be pulsed or modulated stimulation carrier frequencies as illustrated above. Thus, there can be a region of average thermal destruction (within zone 38) and a region of electromagnetic, magnetic, or electronic modification (in the shell between 3800 and 1400) as illustrated in FIG. 13.

If generator 1400 in FIG. 13 produces a pulsed carrier frequency signal, then the peak RF voltages, intensities, power, and currents can be higher than for a continuous wave signal generator that produces a similar average thermal distribution which is, for example, the same size as isotherm 3800. This difference in signal intensities and electronic qualities of the fields for pulsed versus continuous waveform outputs can produce different clinical results and different tissue function modifications.

FIG. 14 shows another embodiment involving cortical C contact electrodes 2100 and 2200, which can be flat area type electrodes placed on the brain surface at strategic positions to produce neural modification within the brain. The connection wire 4000a to generator 1400 supplies the high frequency signal to the electrodes 2100 and 2200. Multiple wires within cable 4600 can give different signals or a bipolar electrode configuration (see the discussion in Cosman's paper on radiofrequency fields) across the electrodes 2100 and 2200. Generator 1400 can also be connected to a catheter or rod-like electrode 4500, which can be placed deep into the brain and have electrode contacts 4000, 4100, and 4200 to produce the electronic signal frequency field effects within the brain nearby. Again, multiple wires can be carried back to generator 1400 through the cable element 4600 for differential signal application on the contacts 4000, 4100, and 4200. Application of the pulsed carrier frequency fields in the configurations such as shown in FIG. 14 can give rise to functional modification of the brain. Alteration of epileptic seizures can be made by application of neuro-modifying, pulsed frequency wave fields in such electrodes. Electrodes such as shown in FIG. 14 are common for recording in the study of epilepsy, as evidenced by brochures available from Radionics, Inc. Their use for modulated frequency application, however, can be applied to alter the brain function near sites where epileptic neural foci are thought to exist. Modification of these epileptic foci can modify or even abolish the epileptic seizure or disease. Similar implantation for application of deep brain or surface-type electrodes on the brain, spinal, cord, or other portions of the body can have similar ameliorating or modifying effects on neural structures or other organs. For example, electrodes such as electrode 4500 can be placed in the thalamus, pallidum, hippocampus, etc. of the brain for alteration, for modification of movement disorders such as Parkinsonism, spasticity, epilepsy, etc.

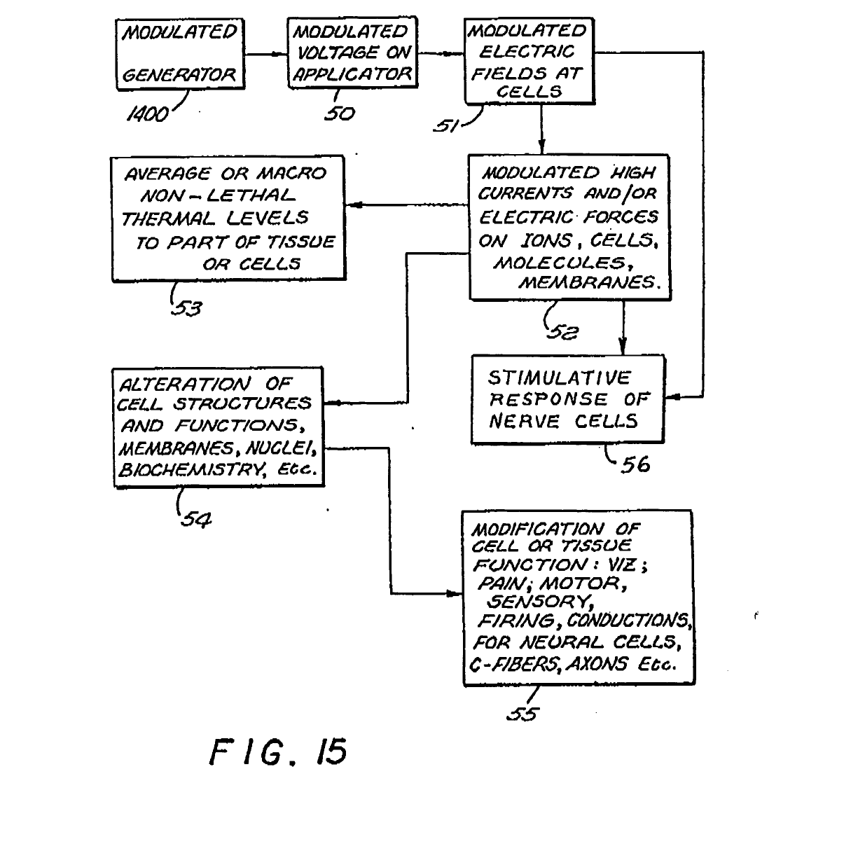

FIG. 15 shows a schematic flow diagram of some ways in which modulated high frequency signals can affect cellular function. Modulated generator 1400 gives rise to a modulated signal output (e.g., voltage) applied to an applicator 50 such as an electrode. This can give rise to modulated electric fields on cells as illustrated by block 51. Electric fields will give rise to electric force or effects within the cells or the tissue (block 52). Electric fields produce alternating electric forces on ions, cell membranes, internal cell structures such as mitochondrion, DNA, etc., or forces of translation and rotation on polar molecules or on membranes having polar internal structures or charged layers. Ionic frictional dissipation effects can occur, producing average or macroscopic thermal elevation (block 53). If average power deposition is low enough, then the average thermal elevations in the tissue near the electrode can be less than 45° C. If power deposition is increased, the average temperature can exceed 45° C. Even at low temperatures (for example 42° C.), electric forces and currents within the cell (block 52) can cause neural modification effects (block 54). Pulsed fields, voltages, or current can act on un-myelinated pain-carrying fibers such as C fibers differently from other more myelinated cells such as A fibers (block 55). The myelin sheath acts as a dielectric or capacitive protective layer on a nerve axon. C fibers, which primarily carry pain sensations, have minimal myelin sheath or no myelin sheath, and thus may be more susceptible to strong pulsed electric fields, currents, or forces, even without significant heating of the nerve tissues.

The action of the modulated signal outputs on neural tissue may eliminate pain while maintaining tactile, sensory, and other neurological functions relatively intact and without some of the deficits, side effects, or risks of conventional continuous-wave heat lesion making. Selectivity by pulsed or modulated fields may arise by selective denervation of pain-carrying structures or cells (such as C fibers) compared to relatively non-destructive modification of other neural structures related to sensation, touch, motor activity, or higher level functions (block 55).

The modulated electric fields, currents, and forces on the neural cellular biostructures (blocks 51 and 52) with one or more carrier frequency components in the PSFR as indicated in block 1400 can produce stimulative response on neural structures (block 56). This can occur simultaneously with the other neural modifications of blocks 53, 54, and 55. The stimulative response 56 and the change in stimulative response 56 can be indicative of the effectiveness of the neural modifications 53, 54, and 55, and can be used to determine the approximate level of signal to be used and the appropriate time to end the signal exposure. This has the advantage that the clinician has a real-time measure of the neural modification process as it happens and by the same agent, the modulated PSFR fields.

In the examples of FIGS. 1 through 15 above, the selection of generator output parameters and the selection of electrode configurations such as size, shape, area, etc., may be interconnected to achieve a neural modification effect without excessive or undesirable heating. At a given average power output of the generator as applied to the electrode adapter, a very small, sharpened electrode may give rise to high current densities in the tissue adjacent to it, which can give rise to focal heating, lesions, thermal cell destruction, cooking, and coagulation of nearby tissue. If the electrode chosen is larger, then such elevated temperature conditions may be reduced as the current density emitting from the electrode is reduced. In a given clinical setting, to achieve the desired neurological modification effect without macroscopic average elevation of neural tissue above, for example, the lesion temperature of approximately 45° C. to 50° C., it may be necessary to select the appropriate parameters for both the lesion generator output such as voltage, current, power, duty cycle, waveform etc., in coordination with the selection of the appropriate electrode geometry (the selection box, for example, being indicated by element 1 of FIG. 1). The system of electronic signal generator combined with the appropriate signal applicator to achieve a given neuro modification can then be considered in combination and cooperation to achieve the effect for a particular clinical site or result.

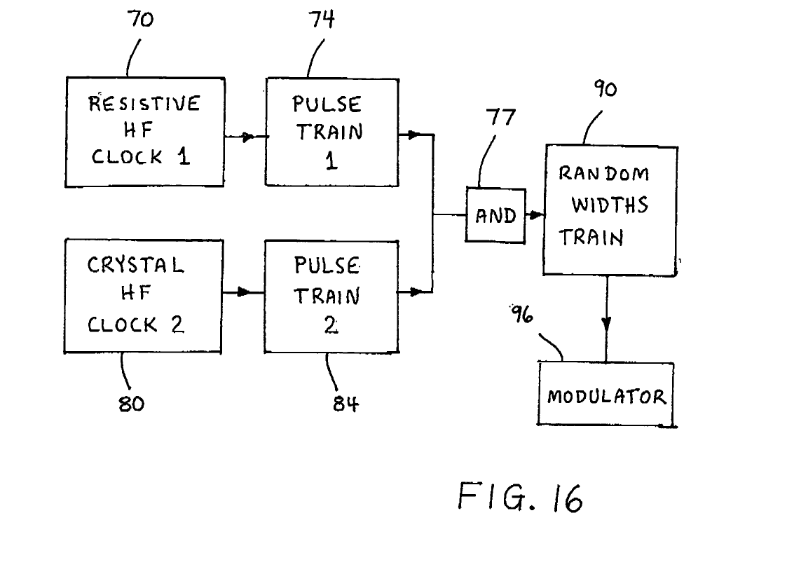

Referring to FIG. 16, one example of a circuit to produce non-predetermined on-time burst durations in a train of regular or constant pulse rate is shown schematically. Clock 1 in block 70 produces a continuous train of pulses 74 at relatively high frequency. It is continuously running, and its clock rate is controlled by resistive devices so that the rate drifts. Its output train 74 is inputted into an “and” switch 77. A crystal clock 2 in block 80 also produces a pulse train 84 with a predetermined rate which is relatively more stable than in block 70. Its output is fed into “and” gate 77. Gate 77 sends out a train of pulses 90, each pulse being started when a pulse from train 84 begins, and each pulse being ended at the trailing edge of a pulse from pulses 74 which overlaps with the pulse from train 84. The output pulse duration from pulses 90 is the sum of pulse width from train 84 plus any residual pulse width from a pulse from pulses 74 which happens to overlap with the pulse from train 84. Clock 70 and clock 80 are in no way synchronized, and 70 drifts in a random way with respect to clock 80. The pulses from pulses 90 will occur at the same constant rate as from train 84, and their widths will be non-predetermined. The pulses 90 can be used to feed into modulator 96 to produce an output signal of modulated carrier waves with bursts of on-time periods having non-predetermined durations.

In view of these considerations, as will be appreciated by persons skilled in the art, implementations and systems should be considered broadly and with reference to the claims set forth below.

I claim:

1. An apparatus for altering a function of living tissue comprising:
an electrode; and
a signal generator coupled to the electrode to generate an electrical signal output to be applied to the living tissue through the electrode, the electrical signal output having an interrupted waveform having bursts of on-time periods of a high frequency carrier frequency component, the on-time periods having non-predictable and uncorrelated time durations, each of the on-time periods being followed by off-time periods, the amplitude of the electrical signal output during the off-time periods being low relative to the amplitude of the electrical signal output of the on-time period, and the bursts of the on-time periods commencing at a regular rate, such that the application of the electrical signal output to the living tissue for a sufficient treatment time will result in alteration of a function of the living tissue.

2. The apparatus of claim 1, wherein the waveform results in an average temperature in the living tissue that is below the lethal temperature range of about 45 to 50° C.

3. The apparatus of claim 1, wherein the waveform results in an average temperature in the living tissue that is above the lethal temperature range of about 45 to 50° C.

4. The apparatus of claim 2, wherein the waveform of the signal output results in peak heating temperatures in the living tissue to be treated during at least some of the on-time periods that are above the lethal temperature range of 45 to 50° C.

5. The apparatus of claim 2, wherein the waveform of the signal output results in peak heating temperatures in the living tissue to be treated during the on-time periods that are below the lethal temperature range of 45 to 50° C.

6. The apparatus of claim 1, wherein the high frequency carrier frequency component comprises at least one radiofrequency wave signal.

7. The apparatus of claim 1, wherein the high frequency carrier frequency component comprises frequency components in the physiologic stimulation frequency range.

8. The apparatus of claim 1, wherein the high frequency carrier frequency component comprises frequency components in the radiofrequency range.

9. The apparatus of claim 1, wherein the on-time periods have random time durations.

10. The apparatus of claim 1, wherein the regular rate is a periodic rate.

11. The apparatus of claim 1, wherein the regular rate is a constant rate.

12. (canceled)

13. A method of altering a function of tissue in a living body comprising:
applying a generated interrupted high frequency waveform having on-time bursts of a high frequency signal output of non-predictable and uncorrelated on-time durations, the high frequency signal output during the on-time bursts comprising at least one high frequency carrier frequency component, each of the on-time bursts being followed by an off-time period of a low signal output relative to the intensity of the preceding on-time period, the off-time periods having a non-predictable and uncorrelated off-time durations, the bursts commencing at a regular rate, to tissue being treated for a treatment time sufficient to result in alteration of a function of the tissue without resulting in an average temperature elevation of the tissue being treated above the lethal temperature range.

14. The method of claim 13, wherein the generated waveform includes bursts generated at a regular rate that is a constant rate.

15. The method of claim 13, wherein the generated waveform includes bursts generated at a periodic rate.

16. (canceled)

17. The method of claim 13, wherein applying included placing an electrode in proximity to a functional target of the nervous system of a patient selected from the group consisting of the spinal cord, spinal ganglia, peripheral nerves, thalamus, basal ganglia of the brain, pallidum, sub-thalamic nucleus, vagus nerve, epilogenic centers, the intervertebral disc, the prostatic nerves, and the cardiac nerves.

18. The method of claim 13, wherein the applying includes applying an electrode adapted to carry the interrupted high frequency waveforms in proximity to a functional target of the nervous system of a patient to cause the patient to experience a reduction in symptoms of a condition selected from the group consisting of epilepsy, tremor, Parkinson's disease, spasticity, mood disorder, cardiac arrhythmia, depression, back pain, spinal pain, cancer pain, urinary disorders associated with the prostate and bladder, headache, and discogenic pain.

19. A method for modification of tissue function comprising:
applying a generated interrupted high frequency waveforms having non-predictable and uncorrelated time periods of on-time bursts of high frequency output followed by non-predictable and uncorrelated off-time periods of low output amplitude relative to the amplitude of the on-time burst, the high frequency signal output during said on-time bursts comprising at least one high frequency carrier frequency component, the rate of on-time bursts being regular, to target tissue to be treated for a treatment time sufficient to result in modification of the target tissue and heating at least a portion of the target tissue during at least a portion of the on-time bursts to temperatures that are above a lethal temperature range for tissue of about 45 to 50° C.

20. The method of claim 19, wherein the generated waveform includes on-time bursts generated at a constant rate.

21. The method of claim 19, wherein the generated waveform includes on-time bursts generated at a periodic rate.

22. An apparatus for alteration of a function of selected tissue comprising:
a signal generator configured to generate interrupted high frequency waveforms having non-predictable and uncorrelated time periods of on-time bursts of high frequency output followed by non-predictable and uncorrelated off-time periods of low output amplitude relative to the amplitude of the on-time burst and the rate of commencing the on-time bursts being regular, the high frequency signal output during said on-time bursts comprising at least one high frequency carrier frequency component; and
an electrode coupled to the generator and being adapted to apply the interrupted high frequency waveforms to the selected tissue, wherein application of the interrupted high frequency waveforms to the selected tissue for a sufficient time alters a function of the selected tissue while inhibiting heating of the selected tissue to lethal temperatures for the selected tissue.

23. An apparatus for altering a function of tissue comprising:
an electrode adapted to apply an amplitude modulated signal to the tissue,
a signal generator adapted to connect to the electrode that generates an electromagnetic signal output with an amplitude modulated waveform having bursts of on-time periods during which a high frequency waveform is applied to the tissue, the high frequency waveform having at least one high frequency component, the bursts of on-time periods having non-predictable and uncorrelated durations and commencing at a regular rate, the amplitude of the waveform during at least some of the bursts of on-time periods being sufficient to elevate the temperature of at least a portion of the tissue to the lethal temperature range during the at least some of the bursts of on-time periods, each burst of on-time period being followed by an off-time period having a non-predictable and uncorrelated off-time duration and during which the amplitude of the waveform is lower than the amplitude of the on-time period, so that when the electromagnetic signal output is applied to the tissue through the electrode for a sufficient treatment time an alteration of a function of the tissue will occur.

24. The apparatus of claim 23, wherein the amplitude of the off-time period is approximately zero.

25. The apparatus of claim 23, wherein the temperature elevation of the tissue during the treatment time on average remains below the lethal temperature range.

26. The apparatus of claim 23, wherein the temperature elevation of at least a portion of the tissue on average exceeds the lethal temperature range for at least some of the treatment time.

27. The apparatus of claim 23, wherein the waveform has an average duty cycle that is a predetermined value, and the duty cycle values for the bursts of on-time periods and the corresponding the following off-time periods vary in a non-predictable variation around the average duty cycle, and the duty cycle values are distributed over a range of values that are non-approximate to the average duty cycle.

28. A method of altering a function of tissue, comprising:
applying an amplitude modulated electrical signal from a signal generator to the tissue through an electrode,
the amplitude modulated electrical signal having a waveform having bursts of electrical signal output for on-time periods, each of the on-time periods being followed by an off-time period of low electrical signal output relative to the electrical signal output of the on-time periods, the waveform during the bursts having at least one high frequency carrier wave oscillation frequency component, the on-time periods of the bursts having non-predictable and uncorrelated durations, and the bursts commencing at a regular rate;
electromagnetically coupling the signal generator to the electrode;
adjusting the amplitude of the signal electrical output for the on-time periods so that during at least some of the bursts the temperature of at least a portion of the tissue is elevated to the lethal temperature range; and
maintaining the application of the amplitude modulated electrical signal to the tissue for sufficient treatment time to alter a function of the tissue.

29. A system for altering a function of tissue of tissue comprising:
a signal applicator adapted to apply an electrical signal output to the tissue;
a signal generator that generates an electrical signal output including high frequency waveforms having non-predictable and uncorrelated time periods of on-time bursts of at least one high frequency carrier wave oscillation frequency component, each on-time burst being followed by an off-time period, the electrical signal output having a strength during the off-time periods being sufficiently low to have no function-altering effect on the tissue, the rate of commencement of the on-time bursts being regular, and the strength of the electrical output signal during the on-time bursts being sufficient to alter the function of the tissue when the signal is applied to the tissue for a sufficient treatment time; and
a signal coupler that couples the signal generator and the signal applicator.

30. A method for altering the function of tissue comprising:
generating an electrical signal including an interrupted high frequency waveform having non-predictable and uncorrelated time periods of on-time bursts of at least one high frequency carrier wave oscillation frequency component, each on-time burst being followed by an off-time period, the strength of the electrical signal during the off-time period being sufficiently low to have no function-altering effect on the tissue, the rate of commencement of the on-time bursts being regular, and the strength of the electrical signal during the on-time bursts being sufficient to alter the function of the tissue when the electrical signal is applied to the tissue for a sufficient treatment time; and
applying the electrical signal to the tissue for the sufficient treatment time.

31. The apparatus of claim 1, wherein the durations of the on-time periods are substantially shorter than durations of the off-time periods.

32. The apparatus of claim 1, wherein the signal amplitude of the electrical signal during the off-periods is reduced substantially relative to the signal amplitude of the electrical signal during the on-periods.

33. The apparatus of claim 1, wherein the signal amplitude of the electrical signal during the off-periods is reduced to substantially zero relative to the signal amplitude of the electrical signal during the on-periods.

34. The apparatus of claim 1, wherein the high frequency carrier frequency component comprises carrier wave oscillation frequencies.

35. The method of claim 13, wherein the durations of the on-time periods are substantially shorter than durations of the off-time periods.

36. The method of claim 13, wherein the signal amplitude of the electrical signal during the off-periods is reduced substantially relative to the signal amplitudes of the electrical signal during the on-periods.

37. The method of claim 13, wherein the signal amplitude of the electrical signal during the off-periods is reduced to substantially zero relative to the signal amplitude of the electrical signal during the on-periods.
